(19) United States
(12) Patent Application Publication
(10) Pub. No.: US 2025/0041427 A1
(43) Pub. Date: Feb. 6, 2025

(54) DINUCLEAR PHOSPHANE GOLD(I) COMPLEXES FOR TREATING CANCER

(71) Applicants: KING FAHD UNIVERSITY OF PETROLEUM AND MINERALS, Dhahran (SA); Imam Abdulrahman Bin Faisal University, Dammam (SA)

(72) Inventors: Adam Ahmed Abdallah SULAIMAN, Dhahran (SA); Anvarhussien A. ISAB, Dhahran (SA); Amany I. ALQOSAIBI, Dammam (SA); Abdul Malik Puthan PEEDIKAKKAL, Dhahran (SA)

(73) Assignees: KING FAHD UNIVERSITY OF PETROLEUM AND MINERALS, Dhahran (SA); Imam Abdulrahman Bin Faisal University, Dammam (SA)

(21) Appl. No.: 18/362,386

(22) Filed: Jul. 31, 2023

(51) Int. Cl. A61K 47/54; A61P 35/00

(52) U.S. Cl. CPC A61K 47/548; A61P 35/00

(57) ABSTRACT

A complex for treating cancer includes a 2-(di-tert-butylphosphino)biphenyl ligand and a bis(diphenylphosphino)alkane ligand. The complex is dinuclear having two gold atoms, wherein the 2-(di-tert-butylphosphino)biphenyl ligand and the bis(diphenylphosphino)alkane ligand are bonded to the gold atoms, and wherein the bis(diphenylphosphino)alkane ligand is bridging the two gold atoms.

### STATEMENT REGARDING PRIOR DISCLOSURE BY THE INVENTORS

Aspects of the present disclosure were described in an article titled “Novel dinuclear gold(I) complexes containing bis(diphenylphosphano)alkanes and (biphenyl-2-yl)(di-tert-butyl)phosphane: synthesis, structural characterization and anticancer activity” published in Issue 35, New Journal of Chemistry on Aug. 1, 2022, which is incorporated herein by reference in its entirety.

### STATEMENT OF ACKNOWLEDGEMENT

The inventors acknowledge the support provided by the Interdisciplinary Research Center for Advanced Materials, King Fahd University of Petroleum and Minerals, Saudi Arabia, through Project INAM2210.

### BACKGROUND

### Technical Field

The present disclosure is directed to gold(I) complexes, and particularly relates to dinuclear phosphane gold(I) complexes and their use in methods for treating cancer.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

The development of anticancer gold(I) complexes bearing phosphane ligands has been an attractive field of research over the past few years. The antirheumatic drug auranofin [(2,3,4,6-tetra-O-acetyl-L-thio-β-D-glyco-pyranosato-S-)(triethylphosphane)gold(I)] has demonstrated remarkable cytotoxic activity in various in vitro and in vivo tumor models, including cisplatin-resistant cell lines. The presence of a thiosugar moiety in auranofin does not contribute to the pharmacological action [T. Marzo, D. Cirri, C. Gabbiani, T. Gamberi, F. Magherini, A. Pratesi, A. Guerri, T. Biver, F. Binacchi, M. Stefanini, A. Arcangeli, L. Messori, Auranofin, Et₃PAuCl, and Et₃PAuI Are Highly Cytotoxic on Colorectal Cancer Cells: A Chemical and Biological Study. ACS Med. Chem. Lett. 2017, 8, 997-1001; T. Marzo, L. Massai, A. Pratesi, M. Stefanini, D. Cirri, F. Magherini, M. Becatti, I. Landini, S. Nobili, E. Mini, O. Crociani, A. Arcangeli, S. Pillozzi, T. Gamberi, L. Messori, Replacement of the Thiosugar of Auranofin with Iodide Enhances the Anticancer Potency in a Mouse Model of Ovarian Cancer. ACS Med. Chem. Lett. 2019, 10, 656-660, incorporated herein by reference in its entirety; and I. Landini, L. Massai, D. Cirri, T. Gamberi, P. Paoli, L. Messori, E. Mini, S. Nobili, Structure-activity relationships in a series of auranofin analogs showing remarkable antiproliferative properties, J. Inorg. Biochem. 2020, 208, 111079].

Et₃PAuCl (Et₃P=triethylphosphane) has demonstrated similar biological properties to auranofin [C. K. Mirabelli, R. K. Johnson, D. T. Hill, L. F. Faucette, G. R. Girard, G. Y. Kuo, C. M. Sung, S. T. Crooke, Correlation of the in vitro cytotoxic and in vivo antitumor activities of gold(I) coordination complexes, J. Med. Chem. 1986, 29, 218-223] indicating that the Et₃PAu⁺ moiety is the “true pharmacophore.” Replacement of the thiosugar ligand of auranofin with a second triethylphosphane molecule led to an increase in antiproliferative activity, indicating that the presence of a second strong and kinetically stable ligand and the resulting cationic nature of the auranofin-derived complex improves cytotoxic potential [I. Landini, L. Massai, D. Cirri, T. Gamberi, P. Paoli, L. Messori, E. Mini, S. Nobili, Structure-activity relationships in a series of auranofin analogues showing remarkable antiproliferative properties, J. Inorg. Biochem. 2020, 208, 111079]. Furthermore, the additional phosphane would make the complex more lipophilic, increasing cellular uptake [H. Scheffler, Y. You, and I. Ott, Comparative studies on the cytotoxicity, cellular and nuclear uptake of a series of chloro gold(I) phosphine complexes, Polyhedron 2010, 29, 66-69]. The cytotoxic potency of complexes against A2780 ovarian carcinoma cells was in the following order: [Au(PEt₃)₂]Cl>[Au(PEt₃)Cl]>[Au(PEt₃)I]>auranofin. Similarly, the cationic complex [Au{P(2,6-dimethoxyphenyl)₃}₂]PF₆ was found to exhibit a 30-fold higher cytotoxicity than cisplatin against prostate (PC-3) and glioblastoma (U87-MG) cancer cells, and also showed the strongest inhibition of spheroid growth in 3D models of cervical (HeLa) cells [T. S. Reddy, S. H. Privér, V. V. Rao, N. Mirzadeh, S. K. Bhargava, Gold(I) and gold(III) phosphine complexes: synthesis, anticancer activities towards 2D and 3D cancer models, and apoptosis-inducing properties. Dalton Trans. 2018, 47, 15312-15323].

The gold(I)-phosphane complexes appear to accumulate in the mitochondria, and, therefore, the mitochondria may represent a site of biological action for the gold(I)-phosphane complexes. The cytotoxic effects of these complexes have been thought to be related to their ability to bind and inhibit the mitochondrial enzyme thioredoxin reductase (TrxR). Moreover, it has been shown that among [Au(PEt₃)Cl], [Au(PEt₃)I], and auranofin, Et₃PAuCl was the most potent cytotoxic agent and the most potent TrxR inhibitory agent [T. Marzo, D. Cirri, C. Gabbiani, T. Gamberi, F. Magherini, A. Pratesi, A. Guerri, T. Biver, F. Binacchi, M. Stefanini, A. Arcangeli, L. Messori, Auranofin, Et₃PAuCl, and Et₃PAuI Are Highly Cytotoxic on Colorectal Cancer Cells: A Chemical and Biological Study. ACS Med. Chem. Lett. 2017, 8, 997-1001]. Furthermore, the mechanisms of anticancer activity of gold complexes have been shown to involve the generation of reactive oxygen species (ROS), the release of cytochrome c, and the subsequent triggering of apoptosis as evidenced by the sub G1 cells accumulation, as well as DNA fragmentation and caspase-3 activation. Gold(I) complexes may induce apoptosis via both intrinsic and extrinsic pathways.

Accumulating support demonstrates that in addition to the bidentate chelating mode of gold(I) complexes, diphosphanes have been applied as bridging ligands in the formation of polymeric chains and macrocyclic complexes. However, the selective formation of mixed phosphane (containing both mono- and diphosphane) gold(I) complexes remain largely unexplored. Caruso et al., reported the antitumor activity of mixed phosphane gold species containing triphenylphosphane (PPh₃) and 1,2-bis(diphenylphosphano)ethane (Dppe) and 1,3-bis(diphenylphosphano)propane (Dppp) [F. Caruso, M. Rossi, J. Tanski, C. Pettinari, F. Marchetti, Antitumor Activity of the Mixed Phosphine Gold Species Chlorotriphenylposphine-1,3-bis(diphenylphosphino)propanegold(I). J. Med. Chem., 2003, 46, 1737-1742; and F. Caruso, C. Pettinari, F. Paduano, R. Villa, F. Marchetti, E. Monti, M. Rossi, J. Med. Chem. 2008, 51, 1584-1591. Chemical Behavior and in Vitro Activity of Mixed Phosphine Gold(I) Compounds on Melanoma Cell Lines]. A complex containing PPh₃ and Dppp was shown to present effective anticancer activity against various tumor cells. An analysis of its activity demonstrates that the PPh₃ unit protects the metal from biological modification [F. Caruso, M. Rossi, J. Tanski, C. Pettinari, F. Marchetti, Antitumor Activity of the Mixed Phosphine Gold Species Chlorotriphenylposphine-1,3-bis(diphenylphosphino)propanegold(I). J. Med. Chem., 2003, 46, 1737-1742]. The cytotoxic activity of [{Au(PPh₃)}₂(μ₂-Dppe)Cl] in JR8, SK-Mel-5, and 2/60 melanoma cell lines is lower than that of [Au(PPh₃)(Dppp)Cl]because of the non-chelating nature of [{Au(PPh₃)}₂(μ₂-Dppe)Cl][F. Caruso, C. Pettinari, F. Paduano, R. Villa, F. Marchetti, E. Monti, M. Rossi, J. Med. Chem. 2008, 51, 1584-1591. Chemical Behavior and in Vitro Activity of Mixed Phosphine Gold(I) Compounds on Melanoma Cell Lines]. The chelation of diphosphane has been shown to contribute to the stability of a species, such as [Au(Dppe)₂]Cl [S. J. Berners-Price, C. K. Mirabelli, R. K. Johnson, M. R. Mattern, F. L. McCabe, L. F. Faucette, C.-M. Sung, S.-M. Mong, P. J. Sadler, S. T. Crooke, Cancer Res. 1986, 46, 5486]. Mitochondria were regarded as the primary targets in apoptosis induced by the mixed phosphine gold species in melanoma cell lines [F. Caruso, R. Villa, M. Rossi, C. Pettinari, F. Paduano, M. Pennati, M. G. Daidone, N. Mitochondria are primary targets in apoptosis induced by the mixed phosphine gold species chlorotriphenylphosphine-1,3-bis(diphenylphosphino)propanegold(I) in melanoma cell lines Zaffaroni, Biochem. Pharmacol. 2007, 73, 773-78].

Although a few gold(I) complexes have been developed in the past, there still exists a need to develop complexes with enhanced lipophilicity and increased cellular uptake. Accordingly, an object of the present disclosure is directed toward the preparation and evaluation of cytotoxic dinuclear phosphane gold(I) complexes including a 2-(di-tert-butylphosphino)biphenyl ligand and/or a bis(diphenylphosphino)alkane ligand for treating cancer with enhanced lipophilicity and cellular uptake.

### SUMMARY

In an exemplary embodiment, a complex is described. The complex includes a 2-(di-tert-butylphosphino)biphenyl ligand and a bis(diphenylphosphino)alkane ligand. The complex is dinuclear having two gold atoms, wherein the 2-(di-tert-butylphosphino)biphenyl ligand and the bis(diphenylphosphino)alkane ligand are bonded to the gold atoms, and wherein the bis(diphenylphosphino)alkane ligand is bridging the two gold atoms.

In some embodiments, the bis(diphenylphosphino)alkane ligand is selected from the group consisting of 1,2-bis(diphenylphosphino)ethane, 1,3-bis(diphenylphosphino)propane, 1,4-bis(diphenylphosphino)butane, and bis[2-(diphenylphosphino)methyl]amine.

In some embodiments, the gold complex further includes a hexafluorophosphate counterion.

In some embodiments, a first phosphorous atom of the 2-(di-tert-butylphosphino)biphenyl ligand, a first gold atom, and a second phosphorous atom of the bis(diphenylphosphino)alkane ligand have a distorted linear geometry with a bond angle from 165° to 178°.

In some embodiments, a half maximal inhibitory concentration of 0.5 to 10 μM in a first cancer cell line, HCT 116.

In some embodiments, the complex has a half maximal inhibitory concentration of 0.5 to 12 μM in a second cancer cell line, MCF-7.

In some embodiments, the complex has a mitochondrial membrane potential of 0.2 to 0.8 in MCF-7 at a complex concentration of 2.5 μM compared to a mitochondrial membrane potential of 1.4 to 1.6 in MCF-7 in the absence of the complex.

In some embodiments, the bis(diphenylphosphino)alkane ligand is bis[2-(diphenylphosphino)methyl]amine.

In some embodiments, the complex has a half maximal inhibitory concentration of 0.6 to 1.2 μM in the first cancer cell line, HCT 116.

In some embodiments, the complex has a mitochondrial membrane potential of 0.2 to 0.5 in MCF-7 at a complex concentration of 2.5 μM compared to a mitochondrial membrane potential of 1.4 to 1.6 in MCF-7 in the absence of the complex.

In some embodiments, the complex has chemical stability in a solution of 100% by volume dimethyl sulfoxide (DMSO), 50% by volume DMSO and 50% by volume water, and 30% by volume DMSO and 70% by volume water based on a total volume of the solution.

In some embodiments, the complex is made by a process including mixing a silver hexafluorophosphate salt in a polar protic solvent with a (biphenyl-2-yl)di-tert-butylphosphane gold chloride salt in a polar aprotic solvent to form a reaction mixture; filtering the reaction mixture to obtain a filtrate; reacting the filtrate with the bis(diphenylphosphino) alkane ligand to form the complex, wherein the silver hexafluorophosphate salt and the (biphenyl-2-yl)di-tert-butylphosphane gold chloride salt are in an amount double that of the molar amount of the bis(diphenylphosphino) alkane ligand.

In an exemplary embodiment, a method for treating cancer is described. The method includes administering the complex to a patient in need of treatment for cancer, wherein during the administering, the complex is contacted with an in vitro cancer cell line.

In some embodiments, the method includes administering the complex induces mitochondrial depolarization and apoptosis in MCF-7.

In some embodiments, the cancer is one or more of breast cancer, adenocarcinoma, breast adenocarcinoma, colon cancer, colorectal cancer, lung cancer, prostate cancer, kidney cancer, liver cancer, melanoma, pancreatic cancer, renal cancer, hepatocellular cancer, cervical cancer, and testicular cancer.

In some embodiments, the method includes administering the gold complex to a subject in a solution containing 0.1 to 150 μM of the gold complex.

In some embodiments, during administering, the complex is contacted with in vitro cancer cell line for 20 to 30 hours.

In some embodiments, the cancer cell line is cultured in a Dulbecco's Modified Eagle Medium (DMEM) with 5 to 15% by weight of a Fetal Bovine Serum (FBS) based on a total weight at 37° C. with 50 to 150 g/mL streptomycin and 50 to 150 units/mL penicillin.

In an exemplary embodiment, a pharmaceutical composition is described. The pharmaceutical composition includes the complex or a pharmaceutically acceptable salt thereof, in combination with a pharmaceutically acceptable carrier, diluent, or excipient.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1 is a flowchart depicting a method of preparing a complex of the present disclosure, according to certain embodiments;

FIG. 2A depicts a chemical structure of complex 1, along with resonance assignment, according to certain embodiments;

FIG. 2B depicts a chemical structure of complex 2, along with resonance assignment, according to certain embodiments;

FIG. 2C depicts a chemical structure of complex 3, along with resonance assignment, according to certain embodiments;

FIG. 2D depicts a chemical structure of complex 4, along with resonance assignment, according to certain embodiments;

FIG. 3 shows an Infrared (IR) spectrum of complex 1 prepared in KBr disks at a resolution of 4.0 cm⁻¹, according to certain embodiments;

FIG. 4 shows IR spectrum of complex 2 prepared in KBr disks at a resolution of 4.0 cm⁻¹, according to certain embodiments;

FIG. 5 shows IR spectrum of complex 3 prepared in KBr disks at a resolution of 4.0 cm⁻¹, according to certain embodiments;

FIG. 6 shows IR spectrum of complex 4 prepared in KBr disks at a resolution of 4.0 cm⁻¹, according to certain embodiments;

FIG. 7 shows proton (¹H) nuclear magnetic resonance (NMR) spectrum of complex 1 in dimethyl sulfoxide-d₆ (DMSO-d₆), according to certain embodiments;

FIG. 8 shows ¹H NMR spectrum of complex 2 in DMSO-d₆, according to certain embodiments;

FIG. 9 shows ¹H NMR spectrum of complex 3 in DMSO-d₆, according to certain embodiments;

FIG. 10 shows ¹H NMR spectrum of complex 4 in DMSO-d₆, according to certain embodiments;

FIG. 11 shows heteronuclear single quantum coherence (HSQC) spectrum for complex 1 in DMSO-d₆, according to certain embodiments;

FIG. 12 shows HSQC spectrum for complex 3 in DMSO-d₆, according to certain embodiments;

FIG. 13 shows carbon-13 (¹³C) NMR spectrum of complex 1 in DMSO-d₆, according to certain embodiments;

FIG. 14 shows ¹³C NMR spectrum of complex 2 in DMSO-d₆, according to certain embodiments;

FIG. 15 shows ¹³C NMR spectrum of complex 3 in chloroform-d (CDCl₃), according to certain embodiments;

FIG. 16 shows ¹³C NMR spectrum of complex 4 in CDCl₃, according to certain embodiments;

FIG. 17 shows phosphorus-31 (³¹P) NMR spectrum of complex 1 in DMSO-d₆, according to certain embodiments;

FIG. 18 shows ³¹P NMR spectrum of complex 2 in DMSO-d₆, according to certain embodiments;

FIG. 19 shows ³¹P NMR spectrum of complex 3 in DMSO-d₆, according to certain embodiments;

FIG. 20 shows ³¹P NMR spectrum of complex 4 in DMSO-d₆, according to certain embodiments;

FIG. 21 is a schematic illustration structural view of complex 1, with an atomic labelling scheme, with displacement ellipsoids drawn at the 15% probability level, according to certain embodiments;

FIG. 22 is a schematic illustration depicting a structural view of the complex 2, with an atomic labelling scheme, showing 15% probability ellipsoids, according to certain embodiments;

FIG. 23 is a schematic illustration depicting a structural view of the complex 4, with an atomic labelling scheme, showing 15% probability ellipsoids, according to certain embodiments;

FIG. 24A shows the effect of the concentrations of complex 1 on the percentage viability of HCT116 cells, according to certain embodiments;

FIG. 24B shows the effect of the concentrations of complex 2 on the percentage viability of HCT116 cells, according to certain embodiments;

FIG. 24C shows the effect of the concentrations of complex 3 on the percentage viability of HCT116 cells, according to certain embodiments;

FIG. 24D shows the effect of the concentrations of complex 4 on the percentage viability of HCT116 cells, according to certain embodiments;

FIG. 24E shows the effect of the concentrations of complex 1 on the percentage viability of MCF-7 cancer cells, according to certain embodiments;

FIG. 24F shows the effect of the concentrations of complex 2 on the percentage viability of MCF-7 cancer cells, according to certain embodiments;

FIG. 24G shows the effect of the concentrations of complex 3 on the percentage viability of MCF-7 cancer cells, according to certain embodiments;

FIG. 24H shows the effect of the concentrations of complex 4 on the percentage viability of MCF-7 cancer cells, according to certain embodiments;

FIG. 25A shows the effect of the concentration of complex 1 on the mitochondria membrane, according to certain embodiments;

FIG. 25B shows the effect of the concentration of complex 2 on the mitochondria membrane, according to certain embodiments;

FIG. 25C shows the effect of the concentration of complex 3 on the mitochondria membrane, according to certain embodiments;

FIG. 25D shows the effect of the concentration of complex 4 on the mitochondria membrane, according to certain embodiments;

FIG. 26A shows ultraviolet-visible (UV-Vis) spectra of (0.1 M, 10 mL) complexes 2 and 3 at an initial time in the mixture of DMSO:H₂O (3:7) and 2′ and 3′ after 24 hours in the same conditions, according to certain embodiments; and

FIG. 26B shows UV-Vis spectra of (0.1 M, 10 mL) complex 4 at an initial time in 100% DMSO and in the mixture of DMSO:H₂O (1:1), 4′ after 24 hours in DMSO, and 4″ after 24 hours in the mixture of DMSO:H₂O (1:1), according to certain embodiments.

### DETAILED DESCRIPTION

In the following description, it is understood that other embodiments may be utilized, and structural and operational changes may be made without departure from the scope of the present embodiments disclosed herein.

Reference will now be made to specific embodiments or features, examples of which are illustrated in the accompanying drawings. In the drawings, whenever possible, corresponding or similar reference numerals will be used to designate identical or corresponding parts throughout the several views. Moreover, references to various elements described herein are made collectively or individually when there may be more than one element of the same type. However, such references are merely exemplary in nature. It may be noted that any reference to elements in the singular may also be constructed to relate to the plural and vice-versa without limiting the scope of the disclosure to the exact number or type of such elements unless set forth explicitly in the appended claims. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “cancer” refers to any one of a large number of diseases characterized by the development of abnormal cells that divide uncontrollably and have the ability to infiltrate and destroy normal body tissue, and may refer to all types of cancer, neoplasm, or malignant tumors found in mammals (e.g., humans), including leukemias, lymphomas, carcinomas, and sarcomas. Exemplary cancers that may be treated with the method or composition provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with the method provided herein include cancer of the thyroid, endocrine system, brain, breast, cervix, colon, head and neck, liver, kidney, lung, ovary, pancreas, rectum, stomach, and uterus. Additional examples include, but are not limited to, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the term “half maximal inhibitory concentration (IC₅₀)” refers to the measure of the potency of a substance in inhibiting a specific biological or biochemical function. IC₅₀ is a quantitative measure that indicates how much of a particular inhibitory substance (e.g., drug) is needed to inhibit, in vitro, a given biological process or biological component by 50%. The biological component can be an enzyme, cell, cell receptor, microorganism, and the like. IC₅₀ values are typically expressed as molar concentration.

As used herein, “analog” and “analogue” refer to a chemical compound that is structurally similar to a parent compound, but differs slightly in composition (e.g., one atom or functional group is different, added, or removed). The analog may or may not have different chemical or physical properties than the original compound and may or may not have improved biological and/or chemical activity. For example, the analog may be more hydrophilic, or it may have altered reactivity as compared to the parent compound. The analog may mimic the chemical and/or biological activity of the parent compound (i.e., it may have similar or identical activity), or, in some cases, may have increased or decreased activity. The analog may be a naturally or non-naturally occurring variant of the original compound. Other types of analogs include isomers (enantiomers, diastereomers, and the like) and other types of chiral variants of a compound, as well as structural isomers.

As used herein, “derivative” refers to a chemically or biologically modified version of a chemical compound that is structurally similar to a parent compound and (actually or theoretically) derivable from that parent compound. A “derivative” differs from an “analog” in that a parent compound may be the starting material to generate a “derivative,” whereas the parent compound may not be used as the starting material to generate an “analog.” A derivative may or may not have different chemical or physical properties of the parent compound. For example, the derivative may be more hydrophilic, or it may have altered reactivity as compared to the parent compound. Derivatization (i.e., modification) may involve the substitution of one or more moieties within the molecule (e.g., a change in a functional group). The term “derivative” also includes conjugates and prodrugs of a parent compound (i.e., chemically modified derivatives that can be converted into the original compound under physiological conditions). A “prodrug” is meant to indicate a compound that can be converted under physiological conditions or by solvolysis to a biologically active compound. Thus, the term “prodrug” refers to a precursor of a biologically active compound that is pharmaceutically acceptable. A prodrug may be inactive when administered to a subject, but is converted in vivo to an active compound, for example, by hydrolysis.

The term “therapeutically effective amount” as used herein refers to the amount of the complex being administered which will relieve to some extent one or more of the symptoms of the disorder being treated. A therapeutically effective amount may be at a level that will exercise the desired effect. In reference to cancer or pathologies related to increased cell division, a therapeutically effective amount refers to that amount which has the effect of at least one of the following: (1) reducing the size of a tumor, (2) inhibiting (that is, slowing to some extent, preferably stopping) aberrant cell division, growth or proliferation, for example, cancer cell division, (3) preventing or reducing the metastasis of cancer cells, (4) relieving to some extent (or, preferably, eliminating) one or more symptoms associated with a pathology related to or caused in part by unregulated or aberrant cellular division, including for example, cancer and (5) inducing apoptosis of cancer cells or tumor cells.

As used herein, the terms “therapies” and “therapy” can refer to any method(s), composition(s), and/or agent(s), and/or complexes that can be used in the prevention, treatment, and/or management of a cancer, disease, or one or more symptoms thereof. Therapy may include immunotherapy, radiation therapy, drug therapy, chemotherapy, combination therapy, and the like.

As used herein, the terms “treat,” “treatment,” and “treating” in the context of the administration of a therapy to a subject in need thereof refer to the reduction or inhibition of the progression and or duration of cancer, the reduction or amelioration of the severity of cancer, and/or the amelioration of one or more symptoms thereof resulting from the administration of one or more therapies. In some embodiments, the subject is a mammalian subject. In one embodiment, the subject is a human. “Treating” or “treatment” of a disease includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer or hyperplasia, these terms simply mean that the life expectancy of an individual affected with cancer will be increased or that one or more of the symptoms of the disease will be reduced. In specific embodiments, such terms refer to one, two, three, or more results following the administration of one, two, three, or more therapies: (1) a stabilization, reduction, or elimination of the cancer stem cell population; (2) a stabilization, reduction, or elimination in the cancer cell population; (3) a stabilization or reduction in the growth of a tumor or neoplasm; (4) an impairment in the formation of a tumor; (5) eradication, removal, or control of primary, regional, and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; and, (12) an increase in the number of patients in remission. In certain embodiments, such terms refer to a stabilization or reduction in cancer stem cell population. In some embodiments, such terms refer to a stabilization or reduction in the growth of cancer cells. In some embodiments, such terms refer to stabilization or reduction in cancer stem cell population and a reduction in the cancer cell population. In some embodiments, such terms refer to a stabilization or reduction in the growth and or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in the survival rate of a patient population. In further embodiments, such terms refer to an increase in the response rate, the durability of response, or the number of patients who respond or are in remission. In some embodiments, such terms refer to a decrease in the hospitalization rate of a patient population and/or a decrease in hospitalization length for a patient population.

A “pharmaceutical composition” refers to a mixture of the compounds described herein or pharmaceutically acceptable salts, esters, or prodrugs thereof, with other chemical components, such as physiologically acceptable carriers and excipients. One purpose of a pharmaceutical composition is to facilitate the administration of at least one gold(I) complex to a subject. A pharmaceutical composition may be formulated to contain a daily dose, or convenient fraction of a daily dose, in a dosage unit. In general, the pharmaceutical composition is prepared according to methods in pharmaceutical chemistry.

“Pharmaceutically acceptable salt” or “pharmaceutically acceptable ester” refers to a compound in a pharmaceutically acceptable form such as an ester, a phosphate ester, a salt of an ester, or a related) which, upon administration to a subject in need thereof, provides at least one of the gold(I) complexes described herein. Pharmaceutically acceptable salts and esters retain the biological effectiveness and properties of the free bases, which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like. Suitable salts include those derived from alkali metals such as potassium and sodium, and alkaline earth metals such as calcium and magnesium, among other acids well-known in the art.

As used herein, a “pharmaceutically acceptable carrier” refers to a carrier or vehicle which may comprise an excipient, diluent, or mixture thereof that does not cause irritation to an organism and does not abrogate the biological activity and properties of the administered gold(I) complex. The term carrier encompasses any excipient, diluent, filler, salt, buffer, stabilizer, solubilizer, lipid, stabilizer, or other material well known in the art for use in pharmaceutical formulations. Suitable formulations may be prepared by methods commonly employed using conventional, organic and inorganic additives, such as an excipient selected from fillers or diluents, binders, disintegrants, lubricants, flavoring agents, preservatives, stabilizers, suspending agents, dispersing agents, surfactants, antioxidants, solubilizers, and the like. The choice of a carrier for use in a composition will depend upon the intended route of administration for the composition. The preparation of pharmaceutically acceptable carriers and formulations containing these materials is described in, for example, Remington's Pharmaceutical Sciences, 21st Edition, ed. University of the Sciences in Philadelphia, Lippincott, Williams & Wilkins, Philadelphia Pa., 2005, which is incorporated herein by reference in its entirety. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

An “excipient” refers to an inert substance added to a pharmaceutical composition to facilitate the administration of a compound further. Examples, without limitation, of excipients include filler or diluents (e.g., sucrose, starch, mannitol, lactose, glucose, cellulose, talc, calcium carbonate, calcium phosphate, and the like), a binder (e.g., cellulose, carboxymethylcellulose, methylcellulose, hydroxy methylcellulose, hydroxypropyl methylcellulose, polypropylpyrrolidone, polyvinylpyrrolidone, gelatin, gum Arabic, polyethylene glycol, starch, and the like), a disintegrants (e.g., sodium starch glycolate, croscarmellose sodium, and the like), a lubricant (e.g., magnesium stearate, light anhydrous silicic acid, talc, sodium lauryl sulfate, and the like), a flavoring agent (e.g., citric acid, menthol, and the like), a preservative (e.g., sodium benzoate, sodium bisulfite, methylparaben, propylparaben, and the like), a stabilizer (e.g., citric acid, sodium citrate, acetic acid, and the like), a suspending agent (e.g., methylcellulose, polyvinyl pyrrolidone, aluminum stearate, and the like), a dispersing agent (e.g., hydroxypropyl methylcellulose and the like), surfactants (e.g., sodium lauryl sulfate, polaxamer, polysorbates, and the like), antioxidants (e.g., ethylene diamine tetraacetic acid (EDTA), butylated hydroxyl toluene (BHT), and the like) and solubilizers (e.g., polyethylene glycols and the like).

Aspects of this disclosure are directed to the development of dinuclear phosphanegold(I) complexes (also referred to as a complex) including bis(diphenylphosphano)alkanes and (biphenyl-2-yl)di-tert-butylphosphane (Bdbp). The synthesized complexes were characterized by Fourier Transform Infrared (FTIR), mass spectrometry (MS), nuclear magnetic resonance (NMR) spectroscopy, and X-ray crystallography. X-ray crystallographic analysis reveals that the complexes exist as dinuclear species. The in vitro cytotoxicity of the complexes was investigated against two established human cancer cell models, colorectal (HCT116) and breast (MCF-7) cancer cells. The four complexes showed cytotoxicity against both cell models with lower IC₅₀ values compared to cisplatin. The mitochondrial membrane potential (ΔΨm) was also assessed to get a better understanding of the underlying molecular mechanism of toxicity. Exposure of MCF-7 cancer cells to the complexes for 24 hours resulted in mitochondrial depolarization (reduced mitochondrial health), thereby potentially triggering the release of cytochrome c and apoptotic cell death. Together, the findings demonstrate that the gold(I) complexes have a greater anticancer activity compared to that of conventional cisplatin, which is potentially mediated through disruption of ΔΨm and induction of apoptosis.

A gold complex for treating cancer is described. The gold complex includes gold or precursors thereof. Suitable examples of gold precursors include, chloro(triethylphosphine)-gold(I), chloro(trimethylphosphine)gold(I), chloro[diphenyl(o-tolyl)phosphine]gold(I), chloro[tri(o-tolyl)phosphine]gold(I), chloro(methyldiphenylphosphine)gold(I), chloro[2-(dicyclohexyl phosphino)-biphenyl]gold(I), chloro[2-di-tert-butyl(2′,4′,6′-triisopropylbiphenyl)phosphine]gold(I), chloro[di(1-adamantyl)-2-dimethylaminophenylphosphine]gold(I), chloro(2-dicyclo hexyl-phosphino-2′-dimethylaminobiphenyl)gold(I), chloro(trimethyl phosphite)gold(I), chloro[(1,1′-biphenyl-2-yl)di-tert-butylphosphine]gold(I), chloro[2-dicyclohexyl(2′,4′,6′-trisopropyl-biphenyl)phosphine]gold(I), chloro[tris(2,3,4,5,6-pentafluorophenyl)-phosphine]gold(I), chloro[tri(p-tolyl)phosphine]gold(I), chloro[2-dicyclohexyl(2′,6′-dimethoxybiphenyl)-phosphine]gold(I), chloro[2-dicyclohexyl(2′,6′;-diisopropoxybiphenyl)-phosphine]gold(I), chloro[2-dicyclohexylphosphino-2′,6′-bis(N,N-dimethylamino)-biphenyl]gold(I), chloro {4-[2-di(1-adamantyl)phosphino]phenylmorpholine}gold(I), chloro(2-di-tert-butylphosphino-3,6-dimethoxy-2′,4′,6′-triisopropylbiphenyl)gold(I), chloro[2-(dicyclohexylphosphino)-3,6-dimethoxy-2′,4′,6′-triisopropyl-1,1′-biphenyl]gold(I), chloro(2-{bis[3,5-bis(trifluoromethyl)-phenyl]phosphino}-3,6-dimethoxy-2′,4′,6′-triisopropyl-1,1′-biphenyl)gold(I), and chloro(2-di-tert-butylphosphino-3,4,5,6-tetramethyl-2′,4′,6′-triisopropylbiphenyl)gold(I). In a preferred embodiment, the gold precursor is chloro[(1,1′-biphenyl-2-yl)di-tert-butylphosphine]gold(I).

The complex further includes two or more ligands, with each ligand that is coordinated, preferably chelated to the gold. The ligands are based on a 2-(di-tert-butylphosphino)biphenyl ligand; and a bis(diphenylphosphino)alkane ligand. The gold complex is dinuclear with two gold atoms. Each gold atom is bonded to both the ligands—namely, the 2-(di-tert-butylphosphino)biphenyl ligand and the bis(diphenylphosphino)alkane ligand. The nature of bonding is such that both the gold atoms in the gold complex are individually bonded to the 2-(di-tert-butylphosphino)biphenyl ligand. A first phosphorous atom of the 2-(di-tert-butylphosphino)biphenyl ligand, a first gold atom, and a second phosphorous atom of the bis(diphenylphosphino)alkane ligand have a distorted linear geometry with a bond angle from 165° to 178°.

The gold complex further includes the bis(diphenylphosphino)alkane ligand, which serves as a bridge binding the two gold atoms. Suitable examples of the bis(diphenylphosphino)alkane ligand include 1,2-bis(diphenylphosphino)ethane, 1,3-bis(diphenylphosphino)propane, 1,4-bis(diphenylphosphino) butane, and bis[2-(diphenylphosphino)methyl]amine. Certain other examples of the bis(diphenylphosphino)alkane ligand may include 1,10-bis(diphenylphosphino)decane, 1,3-bis-(diphenylphosphino)-2-methylpropane, 1,3-bis(diphenylphosphino)-1-butylpropane, 1,3-bis(diphenylphosphino)-2,2-dimethylpropane, 1,2-bis(diphenylphosphino)benzene, and the like. In a preferred embodiment, the bis(diphenylphosphino)alkane ligand is bis[2-(diphenylphosphino)methyl]amine.

The complex of the present disclosure has a half maximal inhibitory concentration of 0.5 to 10 μM in a first cancer cell line, HCT 116, and 0.5 to 12 μM in a second cancer cell line, MCF-7. The complex of the present disclosure has a mitochondrial membrane potential of 0.2 to 0.8 in MCF-7 at a complex concentration of 2.5 μM compared to a mitochondrial membrane potential of 1.4 to 1.6 in MCF-7 in the absence of the complex. The half-maximal inhibitory concentration values change based on the choice of the ligand. For example, when the bis(diphenylphosphino)alkane ligand is bis[2-(diphenylphosphino)methyl]amine, the complex has a half maximal inhibitory concentration of 0.6 to 1.2 μM in the first cancer cell line, HCT116, and a half maximal inhibitory concentration of 0.7 to 1.6 μM in the second cancer cell line, MCF-7. The complex with the bis(diphenylphosphino)alkane ligand being bis[2-(diphenylphosphino)methyl]amine has a mitochondrial membrane potential of 0.2 to 0.5 in MCF-7 at a complex concentration of 2.5 μM compared to a mitochondrial membrane potential of 1.4 to 1.6 in MCF-7 in the absence of the complex.

In certain embodiments, the complex can further include a counter-anion to form a pharmaceutically acceptable salt. Non-limiting examples of counter-anions include halides such as fluoride, chloride, bromide, iodide; nitrate; sulfate; phosphate; methane sulfonate; ethane sulfonate; p-toluenesulfonate, salicylate, malate, maleate, succinate, tartrate; citrate; acetate; perchlorate; trifluoromethanesulfonate (triflate); acetylacetonate; hexafluorophosphate; and hexafluoroacetylacetonate. In some embodiments, the counter-anion is a hexafluorophosphate.

In an embodiment, the gold complex of the present disclosure has a chemical stability in a solution that is greater than 30%, preferably 40%, preferably 50%, preferably 60%, preferably 70%, preferably 80%, preferably 90%, preferably 95%, preferably 97%, preferably 98%, preferably 99%, and most preferably about 100% by volume dimethyl sulfoxide (DMSO). In some embodiments, the gold complex has a chemical stability in a solution that is greater than 50%, preferably 60%, and preferably about 70% water by volume and 30%, preferably 40%, and preferably about 30% DMSO by volume based on a total volume of the solution. In some embodiments, the bis(diphenylphosphino)alkane ligand is bis[2-(diphenylphosphino)methyl]amine and the complex has a chemical stability in a solution of 100% by volume dimethyl sulfoxide (DMSO) and a chemical stability in a solution of 50% by volume DMSO and 50% by volume water based on a total volume of the solution.

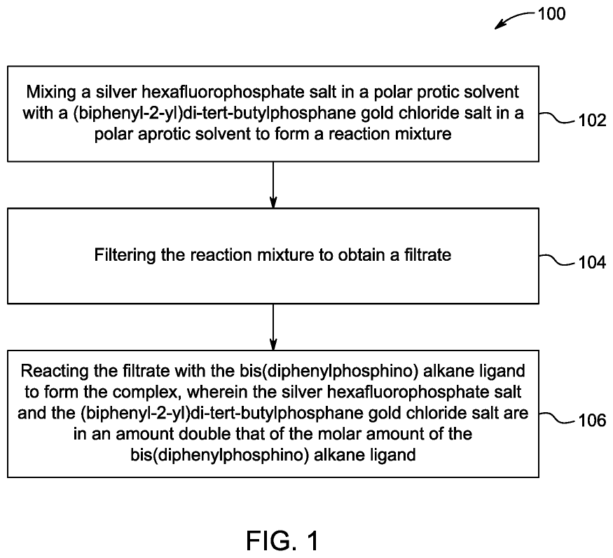

FIG. 1 illustrates a flow chart of a method 100 of preparing the complex. The order in which the method 100 is described is not intended to be construed as a limitation, and any number of the described method steps can be combined in any order to implement the method 100. Additionally, individual steps may be removed or skipped from the method 100 without departing from the spirit and scope of the present disclosure.

At step 102, the method 100 includes mixing a silver hexafluorophosphate salt in a polar protic solvent with a (biphenyl-2-yl)di-tert-butylphosphane gold chloride salt in a polar aprotic solvent to form a reaction mixture. The silver hexafluorophosphate salt and the (biphenyl-2-yl)di-tert-butylphosphane gold chloride salt are mixed in a molar ratio of 1:5 to 5:1, preferably 4:1 to 1:4, preferably 3:1 to 1:3, preferably 2:1 to 1:2, and more preferably about 1:1. Suitable examples of polar protic solvents include, alcohol, water, hydrogen fluoride, formic acid, acetic acid, ammonia, and the like. The alcohol may be ethanol, methanol, isopropanol, and the like. In a preferred embodiment, the polar protic solvent is ethanol. Suitable examples of polar aprotic solvents include, for example, DMF (dimethylformamide), DMPU (dimethyl pyrimidinone), DMSO (dimethyl sulfoxide), DMA (dimethylacetamide), NMP (N-methylpyrrolidone), DMAC (dimethyl acetamide), tetrahydrofuran (THF), acetonitrile, acetone, dichloromethane (DCM), the like, and combinations thereof. In a preferred embodiment, the polar aprotic solvent is DCM. The mixing is carried out at a temperature range of 20-35° C., preferably 20-25° C., and more preferably about 20-22° C., for a period of 10-60 minutes, preferably 20-50 minutes, preferably 25-45 minutes, preferably 30-40 minutes, and more preferably about 30 minutes.

At step 104, the method 100 includes filtering the reaction mixture to obtain a filtrate. The reaction mixture was further filtered to obtain the filtrate. The reaction mixture may be filtered via gravity filtration, vacuum filtration, pressure filtration, and the like through a filter paper, a glass frit, a column, and the like.

At step 106, the method 100 includes reacting the filtrate with the bis(diphenylphosphino) alkane ligand to form the complex. Suitable examples of the bis(diphenylphosphino) alkane ligand include 1,2-bis(diphenylphosphino)ethane, 1,3-bis(diphenylphosphino)propane, 1,4-bis(diphenylphosphino)butane, and bis[2-(diphenylphosphano)methyl]amine. In some embodiments, the molar ratio of the bis(diphenylphosphino) alkane ligand to the (biphenyl-2-yl)di-tert-butylphosphane gold chloride salt are in a ratio range of 1:5 to 5:1, preferably 4:1 to 1:4, preferably 3:1 to 1:3, preferably 2:1 to 1:2, and more preferably about 1:2. In a preferred embodiment, the silver hexafluorophosphate salt and the (biphenyl-2-yl)di-tert-butylphosphane gold chloride salt are in an amount double that of the molar amount of the bis(diphenylphosphino) alkane ligand. The filtrate and the bis(diphenylphosphino) alkane ligands were stirred for a period of 10-60 minutes, preferably 20-50 minutes, preferably 25-45 minutes, preferably 30-40 minutes, and more preferably about 30 minutes and filtered. The colorless product-containing solutions are capped, left undisturbed, and allowed to crystallize for a period of 1-10 days, preferably 3-5 days, to obtain crude white and yellowish-white products. The crude products are further purified by various purification techniques conventionally known in the art (e.g., crystallization, recrystallization, filtration, distillation, and the like) to form the complex.

Another aspect of the present disclosure relates to a pharmaceutical composition comprising one or more of the complexes described herein. The complex described herein, or analogs or derivatives thereof, can be provided in a pharmaceutical composition. Depending on the intended mode of administration, the pharmaceutical composition can be in the form of solid, semi-solid, or liquid dosage forms, such as, for example, tablets, suppositories, pills, capsules, powders, liquids, or suspensions, preferably in a unit dosage form suitable for single administration of a precise dosage. The compositions will include a therapeutically effective amount of one or more of the gold complexes described herein or derivatives thereof in combination with a pharmaceutically acceptable carrier and, in addition, may include other medicinal agents, pharmaceutical agents, carriers, diluents, excipients, or other non-active ingredients. By pharmaceutically acceptable, it is meant a material that is not biologically or otherwise undesirable, which can be administered to an individual along with the selected compound without causing unacceptable biological effects or interacting in a deleterious manner with the other components of the pharmaceutical composition in which it is contained.

The neoplastic activity of the tumor or cancer cells may be localized or initiated in one or more of the following: blood, brain, bladder, lung, cervix, ovary, colon, rectum, pancreas, skin, prostate gland, stomach, breast, liver, spleen, kidney, head, neck, testicle, bone (including bone marrow), thyroid gland, central nervous system.

A pharmaceutical composition including the complex of the present disclosure can then be administered orally, systemically, parenterally, by inhalation spray, rectally, or topically in dosage unit formulations containing conventional nontoxic pharmaceutically acceptable carriers, adjuvants, and vehicles as desired. In some embodiments, the method of administration of the steroid or an analog or derivative thereof is oral. In other embodiments, the compound or an analog or derivative thereof is administered by injection, such as, for example, through a peritumoral injection.

Topical administration can also involve the use of transdermal administration, such as transdermal patches or iontophoresis devices. The term parenteral, as used herein, includes intravesical, intradermal, transdermal, subcutaneous, intramuscular, intralesional, intracranial, intrapulmonary, intracardial, intrasternal, and sublingual injections, or infusion techniques. Formulation of drugs is discussed in, for example, Hoover, John E., Remington's Pharmaceutical Sciences, Mack Publishing Co., Easton, Pa.; 1975. Another example includes Liberman, H. A. and Lachman, L., Eds., Pharmaceutical Dosage Forms, Marcel Decker, New York, N.Y., 1980, which is incorporated herein by reference in its entirety.

Injectable preparations, for example, sterile injectable aqueous or oleaginous suspensions, can be formulated according to the known art using suitable dispersing or wetting agents and suspending agents. The sterile injectable preparation can also be a sterile injectable solution or suspension in a nontoxic, parenterally acceptable diluent or solvent, for example, as a solution in 1,3-butanediol. Among the acceptable vehicles and solvents that can be employed are water, Ringer's solution, isotonic sodium chloride solution, and the like. In addition, sterile, fixed oils are conventionally employed as a solvent or suspending medium. For this purpose, any fixed oil can be employed, including synthetic mono- or diglycerides. In addition, fatty acids, such as oleic acid, find use in the preparation of injectables. Dimethyl acetamide, surfactants including ionic and non-ionic detergents, and polyethylene glycols can be used. Mixtures of solvents and wetting agents, such as those discussed above, are also useful. Suppositories for rectal administration of the compound or an analog or derivative thereof can be prepared by mixing the steroid or an analog or derivative thereof with a suitable non-irritating excipient such as cocoa butter, synthetic mono- di- or triglycerides, fatty acids, and polyethylene glycols that are solid at ordinary temperatures but liquid at the rectal temperature and will therefore melt in the rectum and release the drug.

Solid dosage forms for oral administration can include capsules, tablets, pills, powders, and granules. In such solid dosage forms, the compounds of this disclosure are ordinarily combined with one or more adjuvants appropriate to the indicated route of administration. If administered orally, a contemplated steroid or an analog or derivative thereof can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the active compound in hydroxypropyl methylcellulose. In the case of capsules, tablets, and pills, the dosage forms can also comprise buffering agents such as sodium citrate, magnesium or calcium carbonate, or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated steroid or an analog or derivative thereof of the present disclosure can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and/or sweetening, flavoring, and perfuming agents. The amount of active ingredient that can be combined with the carrier materials to produce a single dosage form varies depending upon the mammalian subject treated and the mode of administration.

Cancers such as, but not limited to, sarcomas, carcinomas, melanomas, myelomas, gliomas, and lymphomas can be treated or prevented with the complex provided herein. In some embodiments, a pharmaceutical composition incorporating the complex of the present disclosure is present in an amount effective for treating a patient having a proliferative disorder selected from the group consisting of head and neck cancer, breast cancer, lung cancer, colon cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), testicular cancer, and the like. In some embodiments, the cancer is preferably one of breast cancer, adenocarcinoma, breast adenocarcinoma, colon cancer, colorectal cancer, lung cancer, prostate cancer, kidney cancer, liver cancer, melanoma, pancreatic cancer, renal cancer, hepatocellular cancer, cervical cancer, testicular cancer, and the like.

The methods for treating cancer and other proliferative disorders described herein inhibit, remove, eradicate, reduce, regress, diminish, arrest, or stabilize a cancerous tumor, including at least one of the tumor growth, tumor cell viability, tumor cell division, and proliferation, tumor metabolism, blood flow to the tumor, and metastasis of the tumor. In some embodiments, after treatment with one or more gold complexes or a pharmaceutical composition thereof, the size of a tumor, whether by volume, weight, or diameter, is reduced by at least 5%, 10%, 15%, 20%, 25%, 30%, 40%, 50%, 60%, 70%, 75%, 80%, 85%, 90%, 95%, 99%, or 100%, relative to the tumor size before treatment. In other embodiments, after treatment with one or more gold complexes of a pharmaceutical composition thereof, the size of a tumor does not reduce but is maintained the same as the tumor size before treatment. Methods of assessing tumor size include, but are not limited to, CT scan, MRI, DCE-MRI, and PET Scan.

In some embodiments, the method for treating cancer and other proliferative disorders involves the administration of a unit dosage or a therapeutically effective amount of the gold complexes or a pharmaceutical composition thereof to a mammalian subject (preferably a human subject) or a patient in need thereof. As used herein, “a subject in need thereof” refers to a mammalian subject, preferably a human subject, who has been diagnosed with, is suspected of having, is susceptible to, is genetically predisposed to, or is at risk of having at least one form of cancer. During administration, the complex is contacted with an in vitro cancer cell line, preferably for 12-36 hours, preferably 15-30 hours, preferably 18-27 hours, preferably 20-24 hours, and preferably about 24 hours. In a preferred embodiment, the cell line is HCT116 or MCF-7. The cancer cell line is cultured in a Dulbecco's Modified Eagle Medium (DMEM) with 5 to 15% by weight of a Fetal Bovine Serum (FBS) based on a total weight at 37° C. with 50 to 150 g/mL streptomycin and 50 to 150 units/mL penicillin. The gold complex is administered to the subject in a solution containing 0.1 to 150 μM of the gold complex. On administration, the complex induces mitochondrial depolarization and apoptosis in MCF-7.

The dosage and treatment durations are dependent on factors such as the bioavailability of a drug, administration mode, toxicity of a drug, gender, age, lifestyle, body weight, the use of other drugs and dietary supplements, cancer stage, tolerance, resistance of the body to the administered drug, and the like, then determined and adjusted accordingly. One or more gold complexes or a pharmaceutical composition thereof may be administered in a single dose or multiple individual divided doses. In some embodiments, the interval of time between the administration of complex or a pharmaceutical composition thereof and the administration of one or more additional therapies may be about 1-5 minutes, 1-30 minutes, 30-60 minutes, 90 minutes, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months, 5 months, 6 months, 7 months, 8 months, 9 months, 10 months, 11 months, 12 months, 1 year, 2 years, 5 years, 10 years, indefinitely, or any period in between. In certain embodiments, the complex and one or more additional therapies are administered less than 1 day, 1 week, 2 weeks, 3 weeks, 4 weeks, 1 month, 2 months, 3 months, 6 months, 1 year, 2 years, or 5 years apart.

In certain embodiments, the complex of the present disclosure or a pharmaceutical composition thereof may be used in combination with one or more other antineoplastic or chemotherapeutic agents. A non-limiting list of examples of chemotherapeutic agents are aflibercept, asparaginase, bleomycin, busulfan, carmustine, chlorambucil, cladribine, cyclophosphamide, cytarabine, dacarbazine, daunorubicin, doxorubicin, etoposide, fludarabine, gemcitabine, hydroxyurea, idarubicin, ifosamide, irinotecan, lomustine, mechclorethamine, melphalan, mercaptopurine, methotrexate, mitomycin, mitoxantrone, pentostatin, procarbazine, 6-thioguanine, topotecan, vinblastine, vincristine, retinoic acid, oxaliplatin, cis-platin, carboplatin, 5-FU (5-fluorouracil), teniposide, amasacrine, docetaxel, paclitaxel, vinorelbine, bortezomib, clofarabine, capecitabine, actinomycin D, epirubicine, vindesine, methotrexate, tioguanine (6-thioguaniue), tipifarnib. Examples for antineoplastic agents which are protein kinase inhibitors include imatinib, erlotinib, sorafenib, sunitinib, dasatinib, nilotinib, lapatinib, gefitinib, temsirolimus, everolimus, rapamycine, bosutinib, pzopanib, axitinib, neratinib, vatalanib, pazopanib, midostaurin and enzastaurin. Examples for antineoplastic agents which are antibodies comprise trastuzumab, cetuximab, panitumumab, rituximab, bevacizumab, mapatumumab, conatumumab, lexatumumab and the like.

### Examples

The disclosure will now be illustrated with working examples, which are intended to illustrate the working of disclosure and not intended to restrictively imply any limitations on the scope of the present disclosure, as many variations thereof are possible without departing from the spirit and scope of the present disclosure. The working examples depict an example of the method of the present disclosure.

Materials and instrumentation Chlorido[(biphenyl-2-yl)di-tert-butylphosphane)]gold(I), 1,2-bis(diphenylphosphano) ethane, 1,3-bis(diphenylphosphano)propane, 1,4-bis(diphenylphosphano)butane, and bis[2-(diphenylphosphano)methyl]amine was purchased from Strem Chemicals Inc. (Newburyport, Massachusetts, United States). AgPF₆ was purchased from Sigma-Aldrich, United States. Ethanol, diethyl ether, acetone, and dichloromethane were obtained from Fluka AG (St. Gallen, Switzerland). All solvents were of analytical grade and were used without further purification.

Elemental analysis was performed on Perkin Elmer Series 11 (CHNS/O), Analyzer 2400. The solid-state Fourier Transform Infrared (FTIR) spectra of the free ligands and their gold(I) compounds were recorded on a Perkin Elmer FTIR 180 spectrophotometer using KBr pellets over the range 4000-400 cm⁻¹ at 4.00 cm⁻¹ resolution. The ¹H, ¹³C, and ³¹P nuclear magnetic resonance (NMR) spectra were measured at 297 K in DMSO-d₆ and CDCl₃ on a JEOL-LA 500 nuclear magnetic resonance (NMR) spectrophotometer, operating at 500.0, 125.65, and 200.0 MHz, respectively, corresponding to a magnetic field of 11.74 T. The spectral conditions for ¹³C NMR were: 32 k data points, 0.967 s acquisition time, 1.00 s pulse delay, and 45° pulse angle. The ¹H and ¹³C spectra were referenced with respect to tetramethylsilane (TMS) as an internal standard. The ³¹P NMR chemical shifts were recorded relative to an external reference (H₃PO₄ in D₂O) at 0.00 ppm.

### Synthesis of Gold(I) Complexes

The complexes were prepared by adding 0.127 g (0.5 mmol) AgPF₆ dissolved in 5.0 mL of ethanol to chlorido[(biphenyl-2-yl)di-tert-butylphosphane)]gold(I) (0.266 g, 0.5 mmol) in 15.0 mL CH₂Cl₂. The mixture was stirred for 30 minutes at room temperature and then filtered. To the collected filtrate, 0.25 mmol of the corresponding ligand: 1,2-bis(diphenylphosphino)ethane, 1,3-bis(diphenylphosphino)propane, 1,4-bis(diphenylphosphino)butane, and bis[2-(diphenylphosphano)methyl]amine, were added. The contents were stirred for an additional 30 minutes and filtered. The clear, colorless solutions were kept in an undisturbed area. After three to five days, white or yellowish-white solids were obtained, which were washed with dichloromethane and diethyl ether three times (5.0 mL). The complexes were then recrystallized from an acetonitrile solution. Suitable crystals of complexes 1, 2, and 4 were chosen for single crystal diffraction analysis.

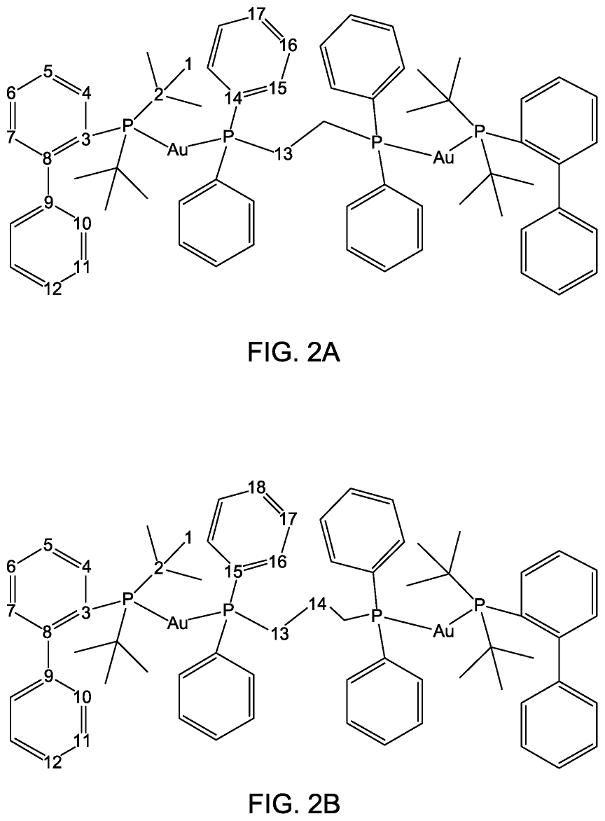

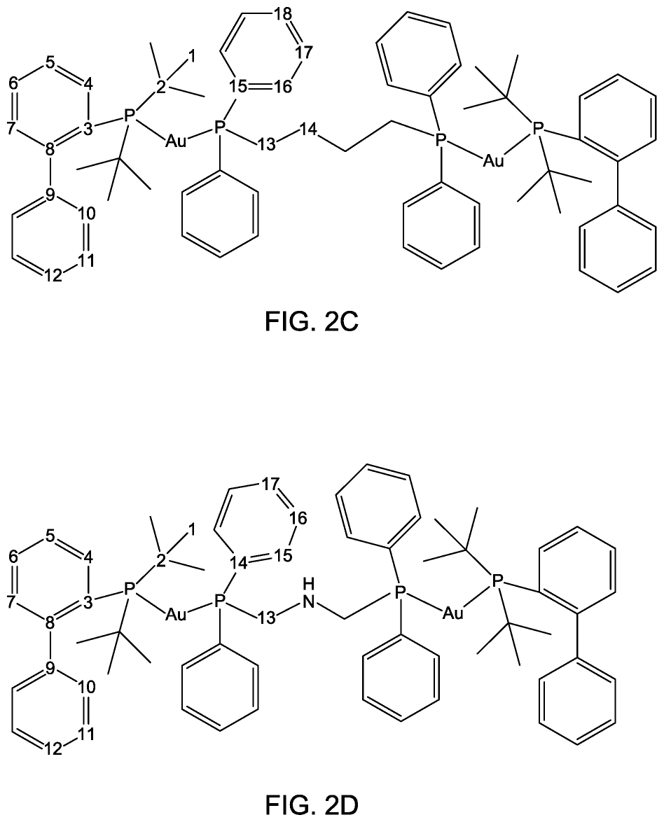

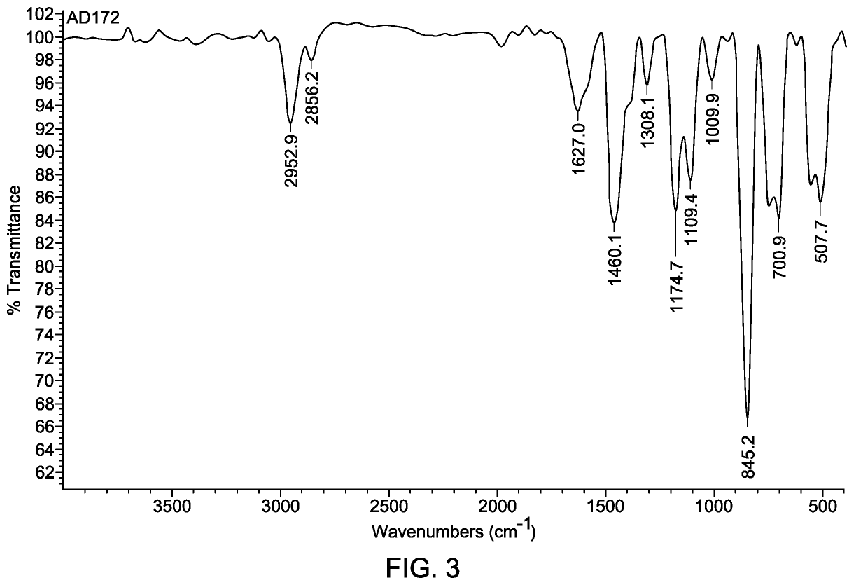

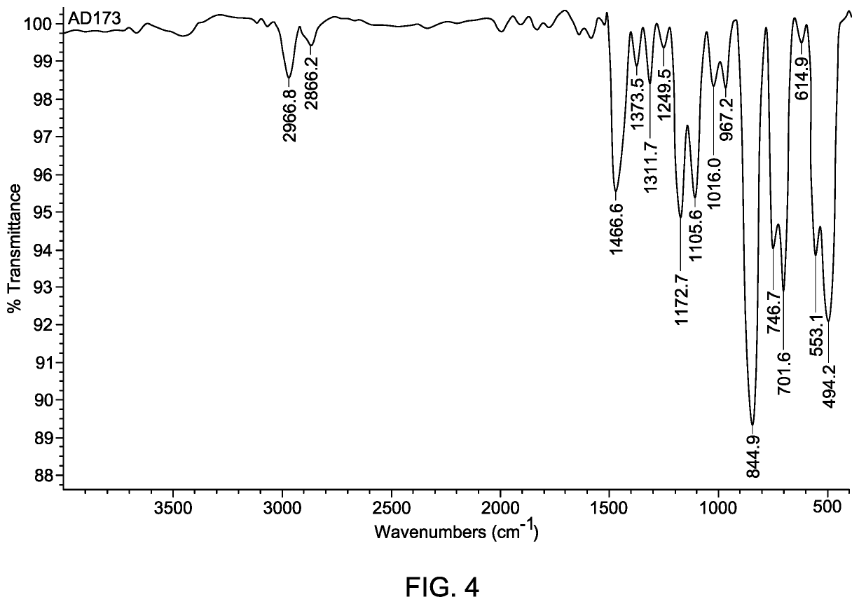

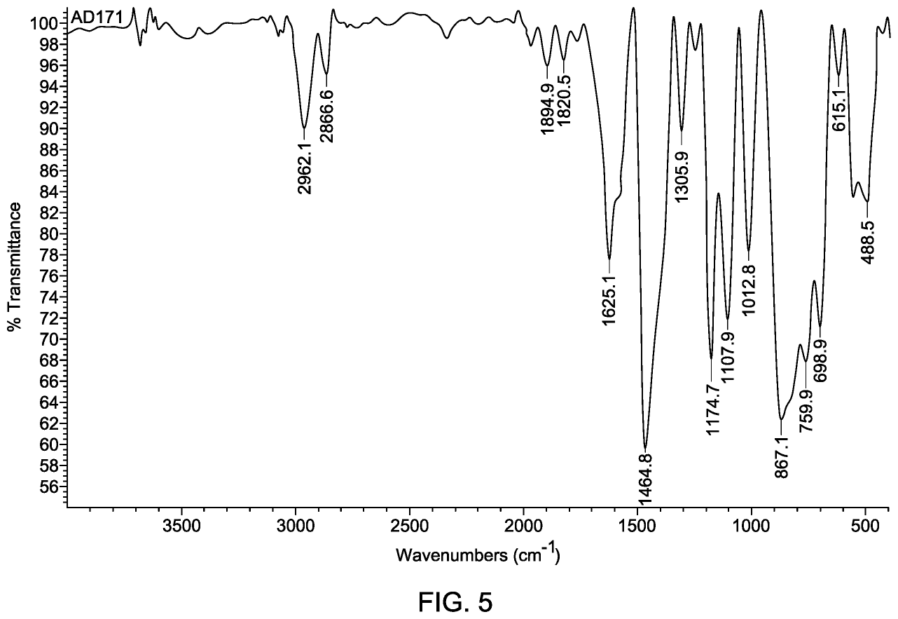

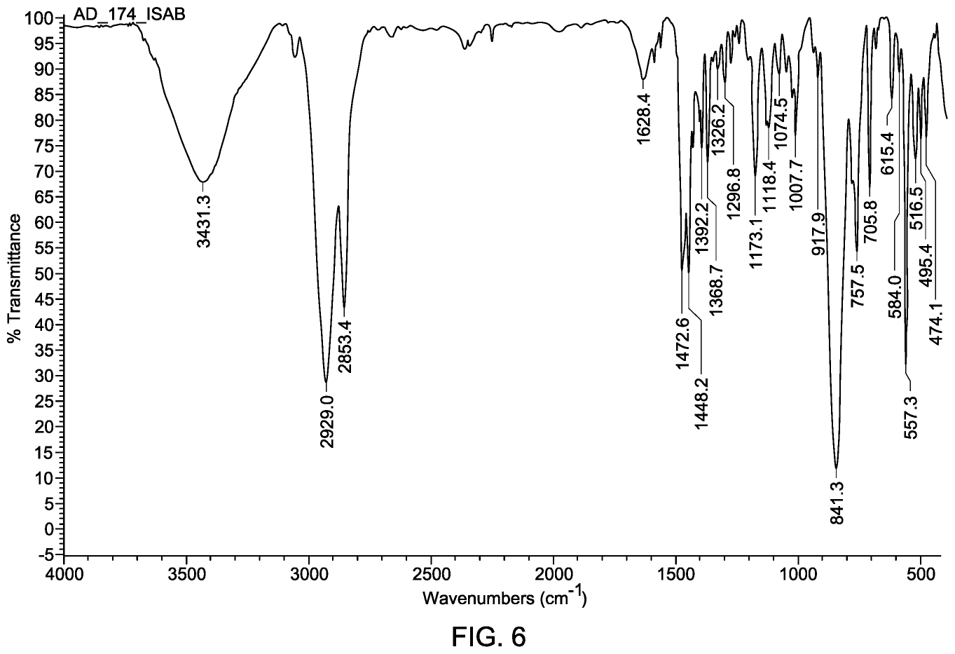

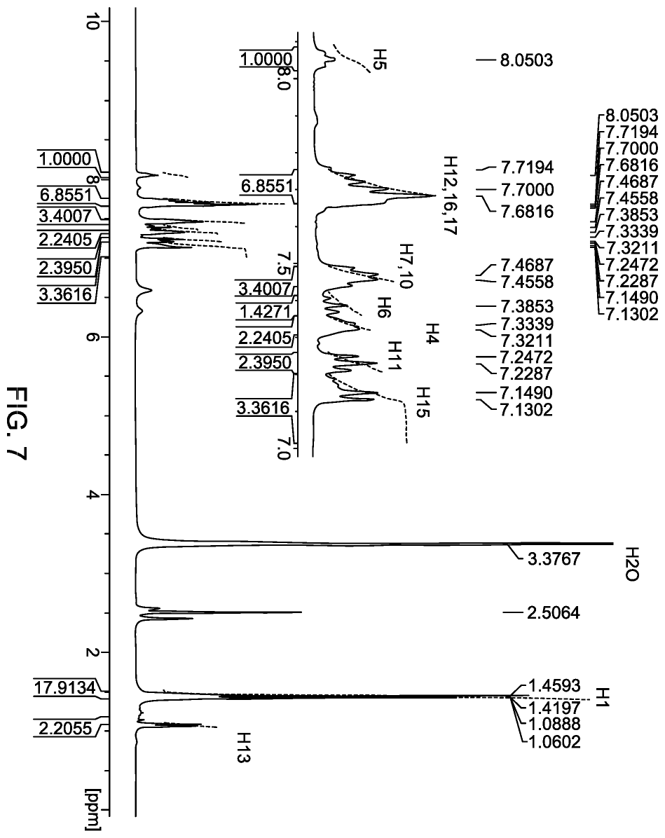

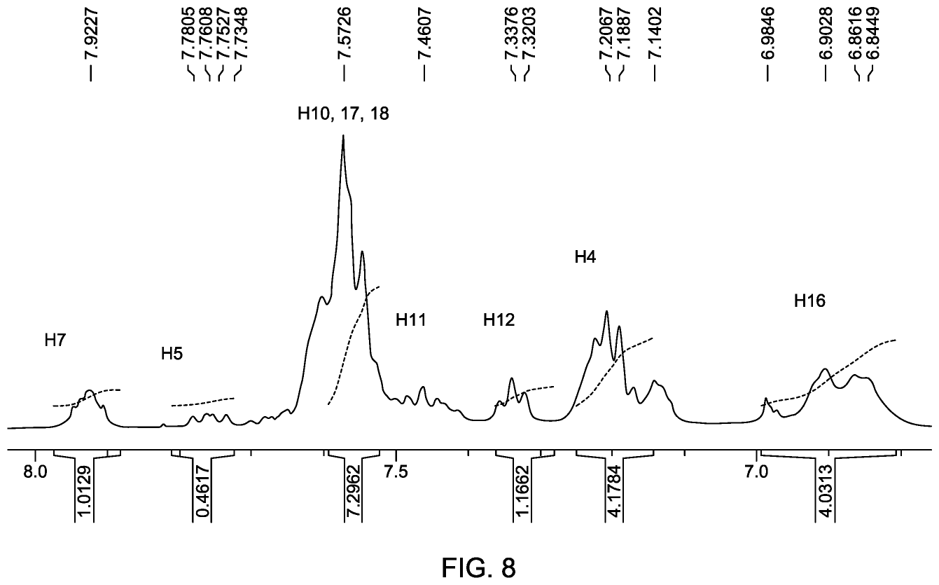

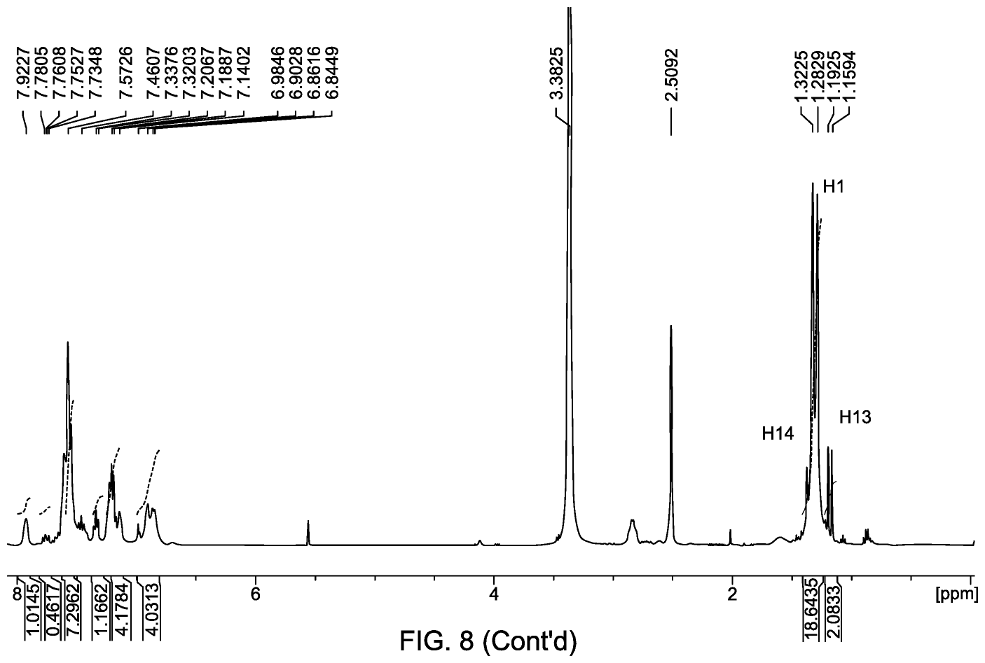

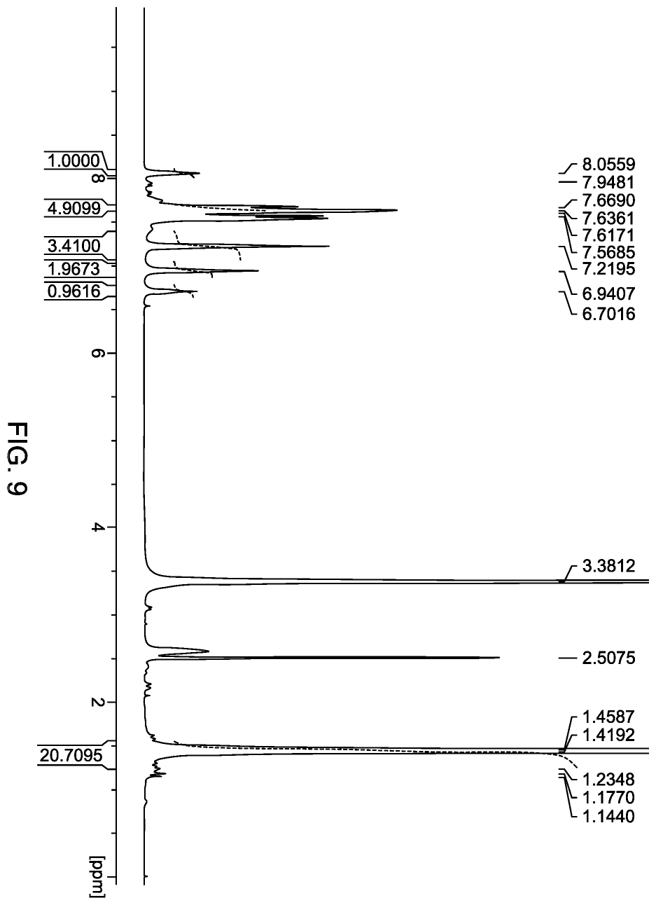

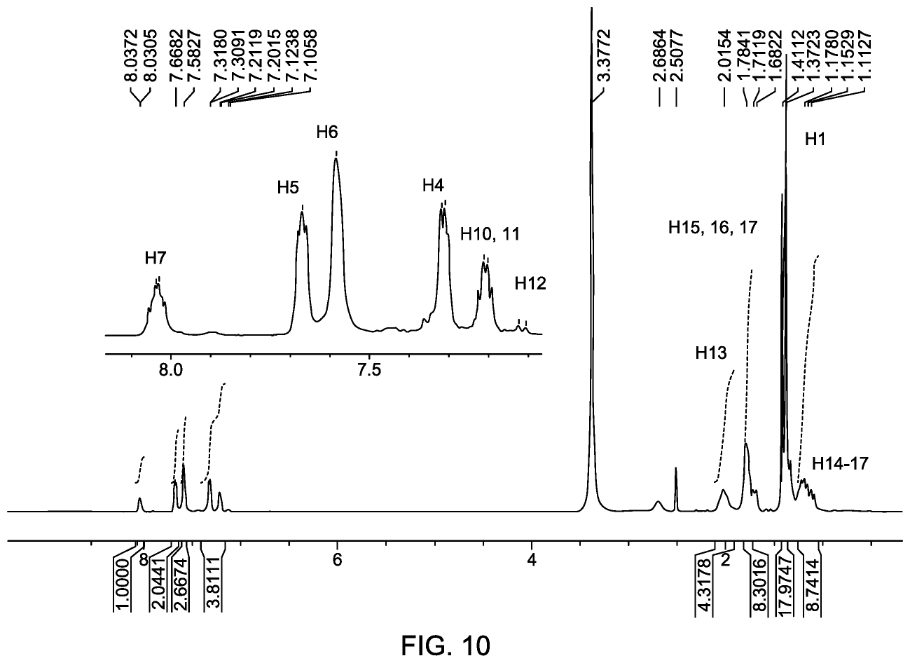

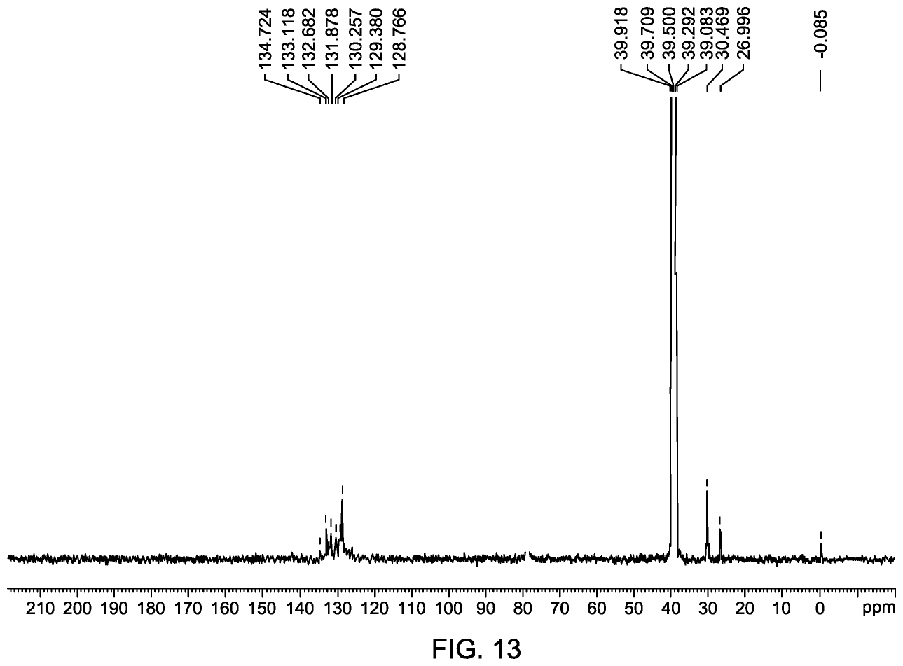

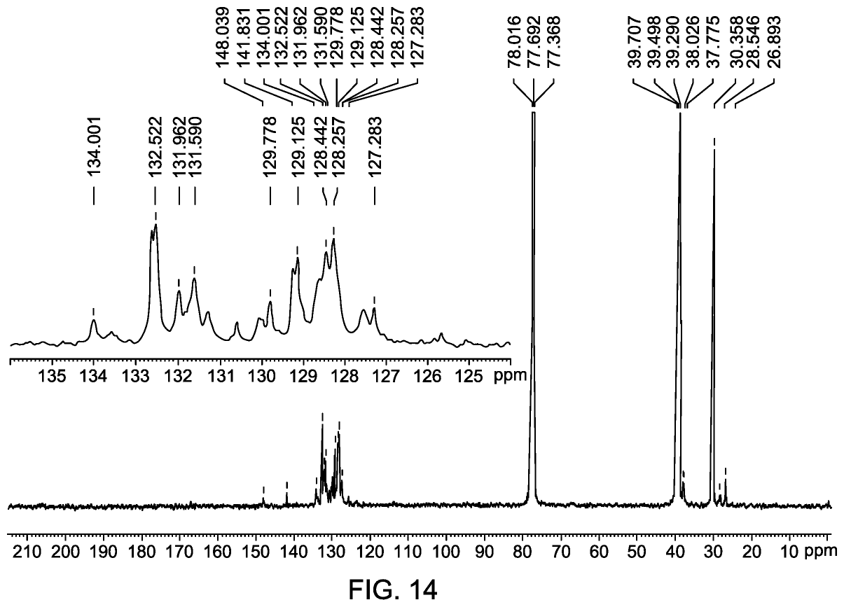

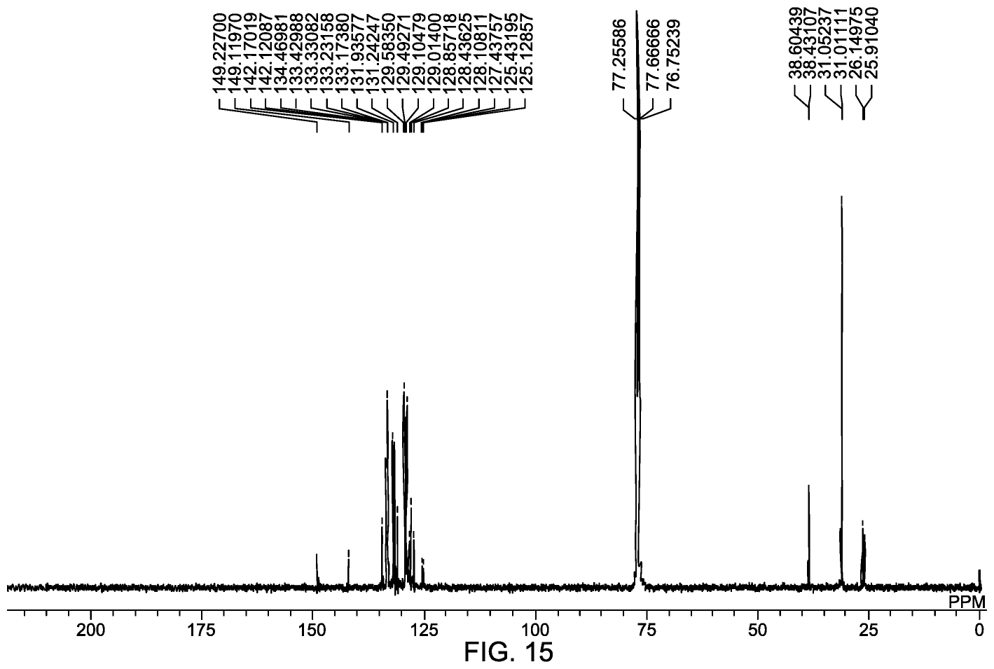

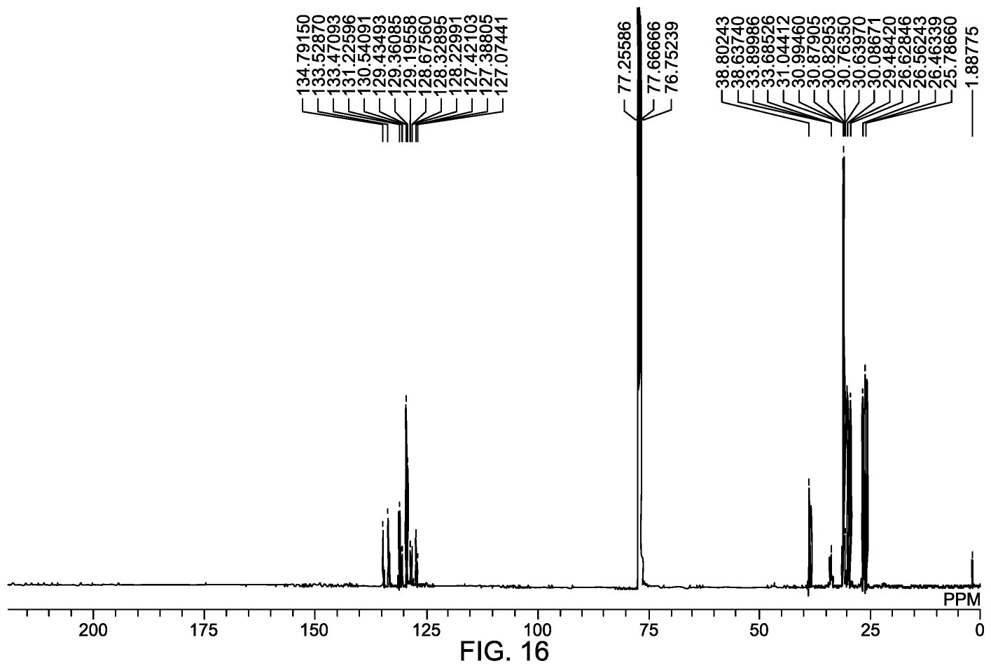

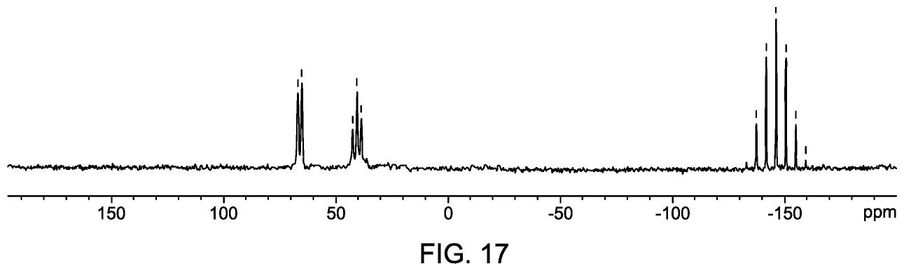

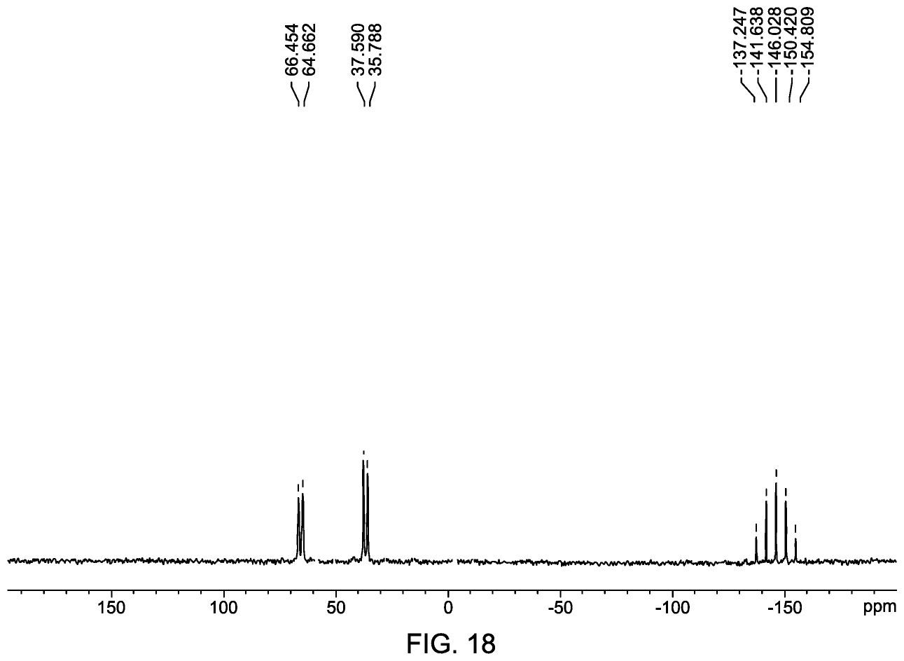

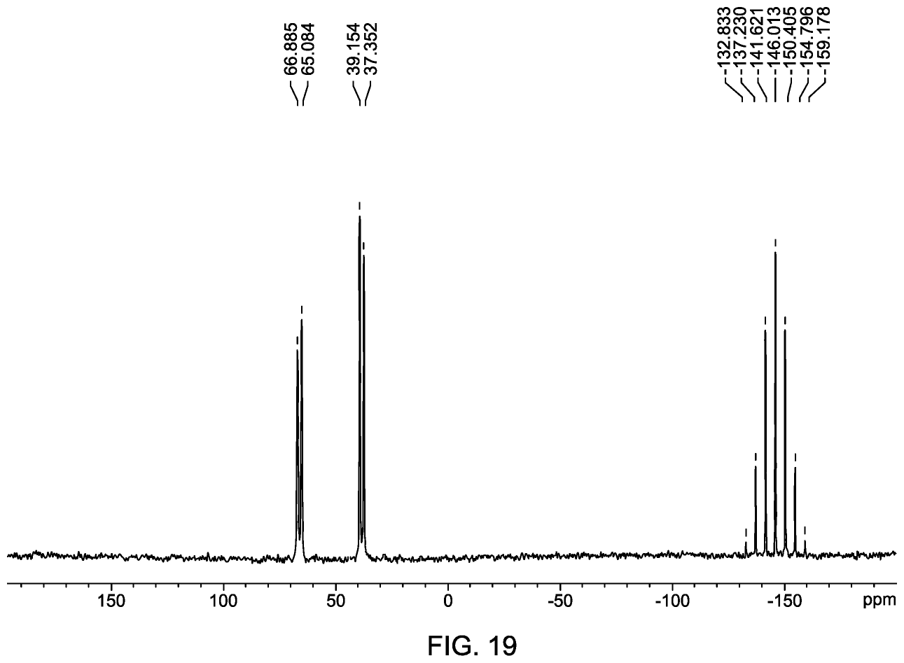

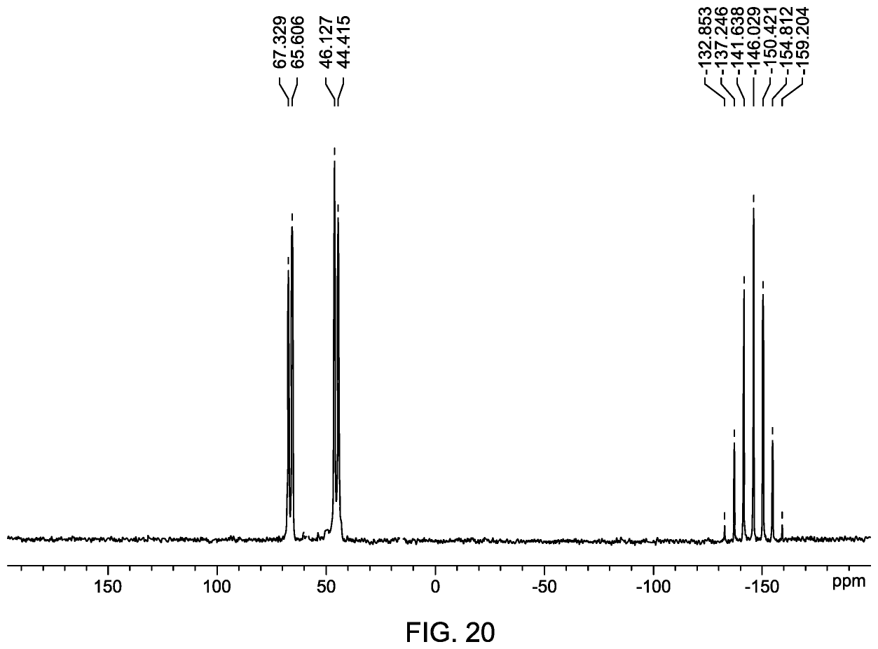

### Analytical and Spectroscopic Data

[Au₂(Bdbp)(Dppe)]P₂F₁₂ (complex 1 (FIG. 2A)), Calc. for C₆₆H₇₈Au₂P₆F₁₂=MW 1679.08 g/mol: C, 47.21; H, 4.68. Found: C, 46.89; H, 4.62. Yield: 0.356 g (81.6%). FTIR (cm⁻¹) (FIG. 3): 2953 v(CH₂) asym, 2856 v(CH₂) sym, 1460 v(C═C), 1175, 1109, 1010 v(C—P), 845 v(P—F), 701 δ(CH₂). ¹H NMR (DMSO-d₆, ppm) (FIG. 7): δ 1.45 (d, J (¹H-³¹P)=15.8 Hz, H1, 36H), 7.32 (dd, H4, 2H), 8.05 (td, H5, 2H), 7.38 (t, H6, 2H), 7.48, (dd, H7, 2H), 7.46 (dd, H10, 4H), 7.22 (t, H11, 4H), 7.71-7.68 (m, H(12, 16, 17), 14H) 1.08 (d, J (¹H-³¹P)=11.4 Hz, H13, 4H), 7.14 (dd, H15, 8H). ¹³C NMR (DMSO-d₆, ppm) (FIG. 13): δ 30.4 C(1), 26.9 C(2), 133.1 C(3), 130.2 C(4), 128.7 C(5), 128.7 C(6), 128.7 C(7), 134.7 C(8), 132.6 C(9), 128.7 C(10), 129.3 C(11), 128.7 C(12), 28.3 C(13), 129.3 C(14), 132.1 C(15), 127.1 C(16), 127.1 C(17). ³¹P NMR (CDCl₃, ppm) (FIG. 17): δ 65.174, 66.932 {²J 351.6}(Bdbp); 38.648, 40.501, 42.399 (Dppe) {Free Dppe=−14.26}; −146.085 (PF₆).

[Au₂(Bdbp)(Dppp)]P₂F₁₂ (complex 2 (FIG. 2B)), Calc. for C₆₇H₈₀Au₂P₆F₁₂=MW 1693.07 g/mol: C, 47.53; H, 4.67. Found: C, 47.12; H, 4.55. Yield: 0.345 g (78.6%). FTIR (cm⁻¹) (FIG. 4): 2967 v(CH₂) asym, 2866 v(CH₂) sym, 1467 v(C═C), 1174, 1106, 1016 v(C—P), 845 v(P—F), 702 δ(CH₂). ¹H NMR (DMSO-d₆, ppm) (FIG. 8): δ 1.32 (d, J (¹H-³¹P)=16.0 Hz, H1, 36H), 7.20 (dd, H4, 2H), 7.92 (m, H5, 2H), 7.75 (td, H6, 2H), 7.76 (dd, H7, 2H), 7.46 (t, H11, 4H), 7.33 (t, H12, 2H), 1.19 (d, J (¹H-³¹P)=13.4 Hz, H13, 4H), 1.37 m (H14, 2H). 6.98-6.84 (dd, H16, 8H), 7.57 (m, H10, 17,18, 12H). ¹³C NMR (DMSO-d₆, ppm) (FIG. 14): δ 30.3 C(1), 37.7 C(2), 28.5 C(13), 26.8 C(14) 127.2-148.04 (aromatic carbons). ³¹P NMR (CDCl₃, ppm) (FIG. 18): δ 64.662, 66.454 {²J 358.4}(Bdbp); 35.788, 37.590 (Dppp) {Free Dppp=−19.08}; −146.028 (PF₆). [Au₂(Bdbp)(Dppb)]P₂F₁₂ (complex 3 (FIG. 2C)), Calc. for C₆₈H8₂Au₂P₆F₁₂=MW 1707.14 g/mol: C, 47.84; H, 4.84. Found: C, 47.25; H, 4.63. Yield: 0.351 g (79.2%). FTIR (cm⁻¹) (FIG. 5): 2962 v(CH₂) asym, 2867 v(CH₂) sym, 1465 v(C═C), 1175, 1108, 1013 v(C—P), 867 v(P—F), 699 (CH₂). ¹H NMR (DMSO-d₆, ppm) (FIG. 9): δ 1.45 (d, J (¹H-³¹P)=15.8 Hz, H1, 36H), 1.17 (d, J(¹H-³¹P)=13.2 Hz H13, 4H), 1.14, 1.17 (H14, 4H), 8.05 (m, H5, 2H), 7.66 (dd, H6, 2H), 7.94 (dd, H7, 2H), 7.63 (d, H10, 4H), 7.56 (td, H11, 4H), 6.94 (t, H12, 2H), 1.19 (d, J (¹H-³¹P)=13.4 Hz, H13, 4H), 1.23 m (H14, 2H), 6.70 (dd, H16, 8H), 7.21 (m, 17,18, 12H). ¹³C NMR (CDCl₃, ppm) (FIG. 15): δ 31.0 C(1), 38.4 C(2), 26.1 C(13), 25.9 C(14), 125.1-149.2 (aromatic carbons). ³¹P NMR (CDCl₃, ppm) (FIG. 19): δ 65.084, 66.885 {²J 360.2}(Bdbp); 37.352, 39.154 (Dppb) {Free Dppb=−17.84}; −146.013 (PF₆).

[Au₂(Bdbp)(Dpma)]P₂F₁₂ (complex 4 (FIG. 2D)), Calc. for C₆₆H₁₀₃Au₂NP₆F₁₂=MW 1718.28 g/mol: C, 46.13; H, 6.04; N, 0.82. Found: C, 45.85; H, 5.95; N, 0.79. Yield: 0.357 g (80.0%). FTIR (cm⁻¹) (FIG. 6): 3431 v(N—H), 2929 v(CH₂) asym, 2853 v(CH₂) sym, 1473 v(C═C), 1448 v(C—N), 1173, 1118, 1008 v(C—P), 841 v(P—F), 706 δ(CH₂) %). ¹H NMR (DMSO-d₆, ppm) (FIG. 10): δ 2.68 (s, N—H), 1.41 (d, J(¹H-³¹P)=15.6 Hz H1, 36H), 7.31 (dd, H4, 2H), 7.66 (td, H5, 2H), 7.58 (H6, t, 2H), 8.03 (H7, dd, 2H), 7.21-7.20 (m, H10, 11, 8H), 7.12 (t, H12, 2H), 2.01 (m, H13, 2H), 1.17 (H14, m, 2H); 1.78, 1.11 (m, H15, 16H); 1.71, 1.15 (m, H16, 16H), 1.68, 1.17 (m, H17, t, 16H). ¹³C NMR (DMSO-d₆, ppm) (FIG. 16): δ 30.99 C(1), 33.68 C(2), 38.89 C(13), 33.80 C(14), 29.43 C(15), 30.81 C(16), 26.65 C(17), 127.0-134.7 (aromatic carbons). ³¹P NMR (CDCl₃, ppm) (FIG. 20): δ 65.606, 67.329 {²J 344.6}(Bdpb); 44.415, 46.127 (Dpma) {Free Dpma=−10.41}; −146.029 (PF₆).

### Single Crystal X-Ray Structure Analysis

Intensity data for complexes 1, 2, and 4 was collected on a Bruker AXS D8 Quest diffractometer using MoKα (λ=0.71073 Å) radiation and was recorded with a Bruker AXS PHOTON II CPAD detector. The data collection was performed at 298 K using ω- and φ-scans. The data was processed using APEX3 Crystal Structure Analysis Package (Bruker AXS). Data collection: APEX3 [Software for Chemical Crystallography v2016.10-0, Bruker AXS Inc.: Madison, WI, 2016], cell refinement: SAINT [Version 8.37A; Bruker AXS Inc: Madison, WI, 2003. Part of the APEX3 v2016.10-0, Bruker AXS Inc.: Madison, WI, 2016], data reduction: SAINT [Version 8.37A; Bruker AXS Inc: Madison, WI, 2003. Part of the APEX3 v2016.10-0, Bruker AXS Inc.: Madison, WI, 2016], absorption correction: SADABS [Version 2016/2; Bruker AXS Inc: Madison, WI, 2003. Part of the APEX3 v2016.10-0, Bruker AXS Inc.: Madison, WI, 2016; Krause, L. Herbst-Irmer, R. Sheldrick G. M.; Stalke D. J. Appl. Cryst., 2015, 48, 3-10, incorporated herein by reference in its entirety], structure solution: direct methods: (SHELXT-2014) [G. M. Sheldrick, SHELXT-Integrated Space-Group and Crystal-Structure Determination. Acta Cryst. 2015, A71, 3-8, incorporated herein by reference in its entirety] and structure refinement: full-matrix least-squares method on F² (SHELXL-2014) [G. M. Sheldrick, A short history of SITELXL, Acta Cryst. 2008, A64, 112-122, incorporated herein by reference in its entirety] using SITELXT as the graphical user interface [Hiibschle, C. B.; Sheldrick, G. M.; Dittrich, B. ShelXle: A Qt graphical user interface for SHTELXL, J. Appl. Crystallogr. 2011, 44, 1281-1284, incorporated herein by reference in its entirety], molecular graphics: program XP (part of the SHTELXT 6.14 program library) [Interactive Molecular Graphics. Part of Program Library for Structure Solution and Molecular Graphics; Bruker AXS, Inc.: Madison, WI, 2000-2013]. The crystal data and the details of data collection and refinement are summarized in Table 1.

TABLE 1
Summary of crystal data and structure refinement for complexes 1, 2, and 4

| | Value | | |
|---|---|---|---|
| Parameter | 1 | 2 | 4 |
| Empirical formula | C₆₆H₇₈Au₂F₁₂P₆ | C₆₇H₈₀Au₂F₁₂P₆ | C₆₆H₁₀₃Au₂F₁₂NP₆ |
| Formula weight | 1679.03 | 1693.07 | 1719.07 |
| CCDC number | 2162609 | 2063121 | 1986650 |
| Crystal symmetry | Triclinic | Monoclinic | Monoclinic |
| Space group | P −1 | C 2/c | C 2/c |
| a, b, c (Å) | 10.4950(5), | 24.3842(11), | 24.428(7), |
| | 12.7517(6), | 11.8498(5), | 11.823(4), |
| | 13.8793(6) | 26.2952(16) | 26.342(11) |
| α, β, γ(°) | 89.712(2), 77.113(2), | 90, | 90, |
| | 75.288(2) | 117.195(2), 90 | 117.286(5), 90 |
| Cell volume (Å³) | 1748.68(14) | 6758.0(6) | 6761 |
| Z | 1 | 4 | 4 |
| Dₓ (g m⁻³) | 1.594 | 1.586 | 1.611 |
| μ (mm⁻¹) | 4.397 | 4.549 | 4.548 |
| F(000) | 830 | 3036 | 3088 |
| Crystal size (mm) | 0.40 × 0.40 × 0.35 | 0.40 × 0.40 × 0.40 | 0.40 × 0.40 × 0.40 |
| Temperature (K) | 296(2) | 296(2) | 296(2) |
| Wavelength (Å) | 0.71073 | 0.71073 | 0.71073 |
| θ range (°) | 2.061-28.358 | 2.892-32.103 | 2.314-34.326 |
| Index ranges | −14:14, −17:17, | −36:36, −17:17, | −38:38, −18:18, |
| | −18:18 | −39:39 | −41:41 |
| Reflections collected | 96674 | 31109 | 35044 |
| Independent reflections | 8718 [R(int) = | 11489 [R(int) = | 9926 [R(int) = |
| | 0.0633] | 0.0691] | 0.0454] |
| Data/restraints/ | 8718/0/395 | 11489/0/400 | 9926/0/400 |
| parameters | | | |
| R[F² > 2σ(F²)], wR(F²), S | 0.0396, | 0.0492, | 0.0506, |
| | 0.0973, 1.105 | 0.1035, 1.023 | 0.1632, 0.973 |
| Aρₘₐₓ, Aρₘᵢₙ (e Å⁻³) | 1.681, −1.254 | 1.562, −1.577 | 0.186, 0.162 |

### Cell Culture

Human colorectal carcinoma cell line HCT116 and human breast adenocarcinoma cell line MCF-7 were purchased from ATCC. The cells were cultured in Dulbecco's Modified Eagle's Medium (DMVEM) complemented with 10% fetal bovine serum (FBS) at 37° C. (5% CO₂ and 95% humidity). 100 g/mL streptomycin and 100 U/mL penicillin were used in the media to prevent microbial growth. Assays were performed on cells from passages 3-8.

### Cell Proliferation Assay

AlamarBlue® was used to determine the drug-dose response according to the manufacturer's instruction (Thermo Fisher, Waltham, Massachusetts, United States). The samples of complexes were diluted to a working concentration in DMVEM before their exposure to the cancer cells. The cancer cells were cultivated in 96-well plates for 24 hours. The cell culture medium was then removed and the diluted test compounds were added to the wells. The well plates were incubated for 24 hours at 37° C. with 5% CO₂. Subsequently, the cell culture medium was removed for the addition of 10% AlamarBlue® (diluted in DMEM) to each well. The plates were incubated for 3 hours at 37° C. protected from light. The cytotoxicity/proliferation was measured using fluorescence spectrophotometry with fluorescence being read at an excitation wavelength of 560 nm and an emission wavelength of 590 nm. To calculate the percentage difference in reduction between treated and control cells in cytotoxicity/proliferation assays, the following formula was used:

$$\% viability = ( ( experimental RFU with chemical compound ) \text{} / ( untreated cell control RFU value ) ) \times 100$$

where RFU stands for relative fluorescence units.

### Mitochondrial Membrane Potential (ΔΨm)

MCF-7 cells (1×10⁴ cells per well in a 96-well plate) were treated with different concentrations of complexes, and untreated cells were used as a negative control. After treatment for 24 hours, 5,5′,6,6′-tetrachloro-1,1′,3,3′-tetraethylbenzimidazolocarbocyanine iodide (JC-1) dye (Thermo Fisher, Waltham, Massachusetts, United States) was added to each well containing the cells. The staining was performed following the protocol provided by the manufacturer. A fluorescence spectrophotometer was used to measure the fluorescence of JC-1-stained cells, and fluorescence signals in the red and green channels were recorded separately.

### Statistical Analysis

GraphPad Prism was used to analyze the data and perform statistical analysis (ANOVA). Data are presented as the mean of three replicates ±standard error. Results were considered significant when P≤0.05. Synthesis and spectroscopic characterization

The chemical structures of complexes 1-4 are provided in FIG. 2A-2D, respectively. The synthesis of complexes 1-4 involved a two-step process. In the first step, the chloride complex, chlorido[(biphenyl-2-yl)di-tert-butylphosphane)]gold(I), was converted into a PF₆ species. In the second step, diphosphane ligands were added to obtain the required complexes. Suitable crystals of 1, 2, and 4 were obtained from acetonitrile solution. The collected products1-4 have the composition [Au₂{(biphenyl-2-yl)di-tert-butylphosphane)}(diphosphane)](PF₆)₂ as indicated by the elemental analysis. The complexes were found as dinuclear ionic species possessing linear geometry at the gold(I) center (vide infra).

The selected FTIR bands of the prepared complexes are listed in the experimental section. The representative FTIR spectra are shown in FIG. 3 to FIG. 6. In the IR spectra of the complexes 1-4, the v(P—C) bands of phosphane ligands were observed around 1100 cm⁻¹ and 1000 cm⁻¹ [A. K. Abogosh, M. K. Alghanem, S. Ahmad, A. Al-Asmari, H. M As Sobeai, A. Alhoshani, A. A. A. Sulaiman, M. Fettouhi, A. A. Isab, A Novel Cyclic Dinuclear Gold(I) Complex Induces Anticancer Activity via Oxidative Stress-Mediated Intrinsic Apoptotic Pathway in MDA-MB-231 Cancer Cells. Dalton Trans. 2022, 15, 2760-2769; and A. A. Sulaiman, A. Alhoshani, H. M. As Sobeai, M. Alghanem, A. K. Abogosh, S. Ahmad, M. Altaf, M. Monim-ul-Mehboob, H. Stoeckli-Evans, A. A. Isab, Anticancer activity and X-ray structure determination of gold(I) complexes of 2-(diphenylphosphanyl)-1-aminocyclohexane, Polyhedron 2020, 183, 114532, both of which are incorporated herein by reference in their entirety]. The v(C═C) modes of the aromatic ring were detected at about 1470 cm⁻¹. The medium intensity bands in the region of 2900 cm⁻¹ in the IR spectra are assigned to v(C—H) (aliphatic) vibrations, while those above 3000 cm⁻¹ represent the aromatic C—H stretches. The absorptions around 700 cm⁻¹ indicate the methylene (CH₂) rock. In complex 4, the v(N—H) and v(C—N) vibrations due to the amino group of the phosphane ligand appeared at 3431 and 1448 cm⁻¹, respectively. A strong band near 840 cm⁻¹ marks the presence of a P—F bond in the PF₆⁻ ion.

The ¹H NMR (FIG. 7 to FIG. 10), ¹³C NMR (FIG. 13 to FIG. 16), and ³¹P NMR (FIG. 17 to FIG. 20) spectral data of the complexes are provided herein. The representative NMR spectra are shown in FIG. 7-FIG. 20. The ¹H NMR spectra of complexes 1-4, as shown in FIG. 7 to FIG. 10, respectively, showed multiplets for aromatic protons in the region of δ 6.70-8.08 ppm and show other resonances around δ 1.06-2.01 ppm for methyl and methylene hydrogen atoms. The ¹³C NMR spectra provide an identification of the complexes by displaying signals for carbon atoms. In ¹³C NMR spectra of complexes, 14 peaks for aromatic carbons of phosphanes were detected in the region of 125-150 ppm. The CH₂ and CH₃ moieties are detected between 25-40 ppm. The carbon atoms attached to phosphorus appeared as doublets due to coupling with ³¹P nuclei. The resonances due to diphosphane phenyl groups are more intense than those without the coupling.

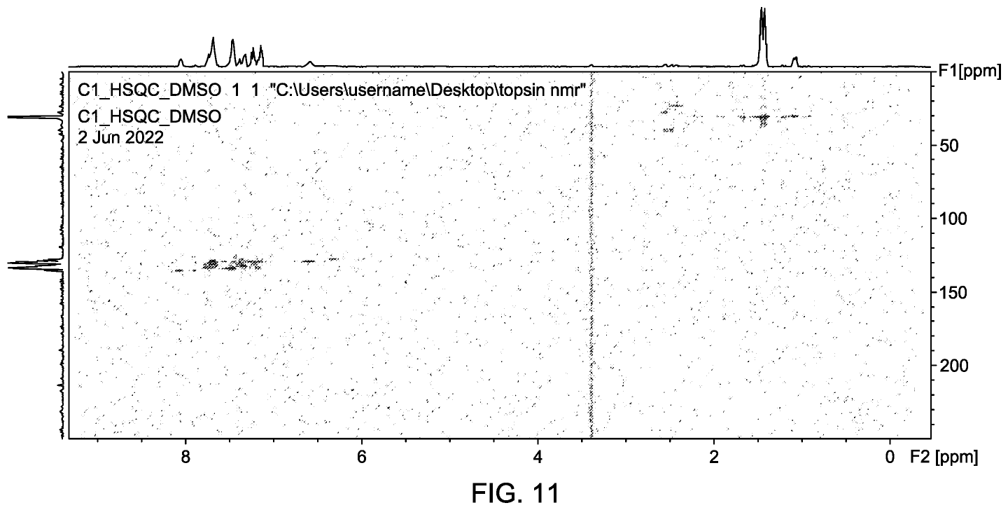

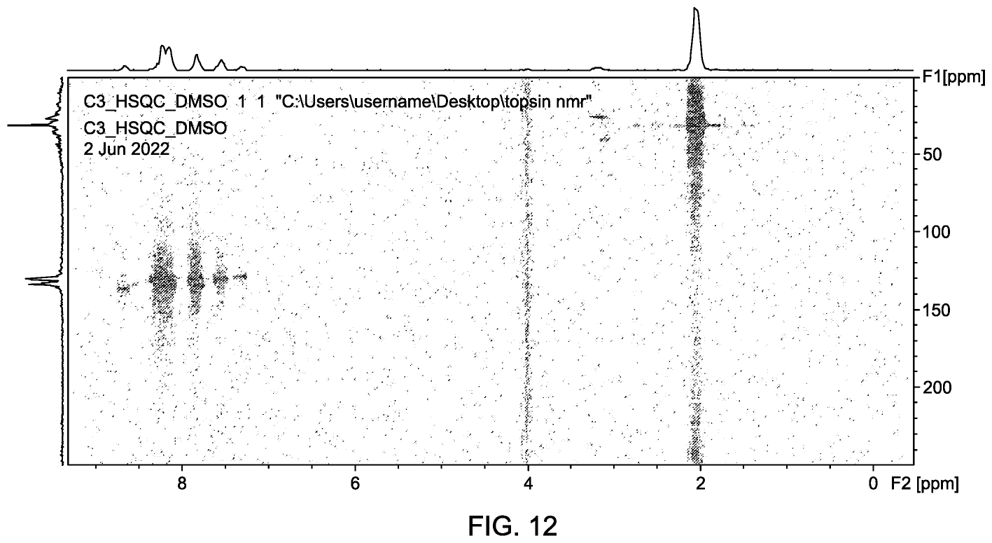

The 2D Heteronuclear Single Quantum Coherence (HSQC)¹³C-¹H direct relationship spectra for complexes 1 and 3 (FIG. 11 & FIG. 12) revealed which hydrogens are attached to which carbons, which is consistent with the 1D ¹H NMR and ¹³C NMR for similar complexes. The spectrum of complex 1 shows that the aliphatic protons at 1.41 and 1.45 ppm are correlated to an aliphatic carbon which appeared at 30.4 ppm, and protons at 1.06, 1.08 were correlated to carbon at 26.9 ppm, while 7.14-8.05 ppm multiplet (aromatic protons) are correlated to a signal at 127-132 ppm (aromatic carbons). In complex 3, the aliphatic protons at 1.41 and 1.45 ppm were correlated to a carbon which appeared at 31.0 ppm, and a 6.70-8.05 ppm multiplet signal (aromatic protons) are correlated to a signal at 125.1-149.2 ppm (aromatic carbons).

The ³¹P NMR spectra (in CDCl₃) of free diphosphanes, a sharp singlet was observed at −20 to −10 ppm. Upon complexation with gold(I), a large downfield shift was observed in these resonances due to the transfer of electron density from phosphorus atoms of diphosphanes to the metal, causing a deshielding effect at phosphorus nuclei [Seerat-ur-Rehman, S. Ahmad, M. A. Choudhary, M. N. Tahir, I. Ali, M. Aslam, M. Fettouhi, A. A. Isab, M. A. Alotaibi, A. I. Alharthi, Polyhedron 2020, 175, 114209, incorporated herein by reference in its entirety]. The ³¹P NMR spectra of the complexes showed three signals that can be assigned to the three different phosphorus environments in the complexes (Dpma, diphosphane, and PF₆). The spectra of the four complexes 1-4, as shown in FIG. 17-FIG. 20, respectively, showed a doublet resonance for Dpma in the region of about δ 65 ppm. The magnitude of the ²J(P—P) coupling constants for these signals is about 350 Hz. The resonances for diphosphane part also appeared as doublets in complexes 2-4, while in the case of 1, it was a triplet. In addition, the septet due to PF₆⁻ counter ion was observed at δ −146 ppm (Table 2).

TABLE 2
³¹P NMR chemical shifts (ppm) for free ligands
(L1-L4) and gold(I) complexes 1-4 in CDCl₃/DMSO-d₆,
where L1-L4 correspond to the diphosphane ligands
used in the synthesis of complexes 1-4, respectively.

| Compound | ³¹P NMR signal (ppm) |
|---|---|
| L1 | −14.26 |
| 1 | 40.50t, 65.17d, −146.08 septate |
| L2 | −19.08 |
| 2 | 35.73d, 64.66d, 146.02 septate |
| L3 | −17.84 |
| 3 | 39.15d, 65.08d, −146.01 septate |
| L4 | −10.41 |
| 4 | 44.41d, 65.60d, 146.02 septate |

### X-Ray Structures of Complexes 1, 2, and 4

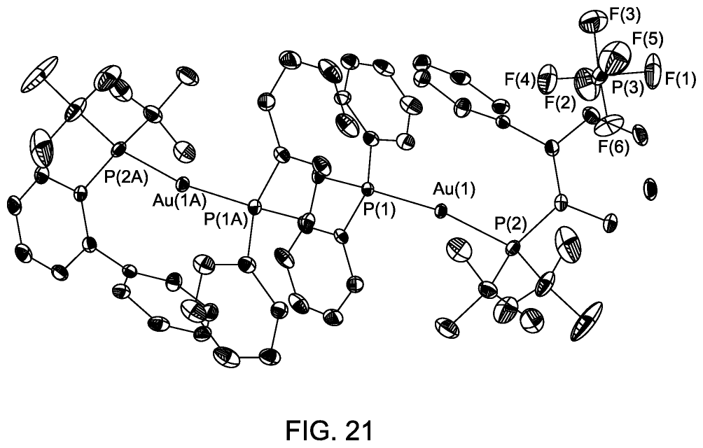

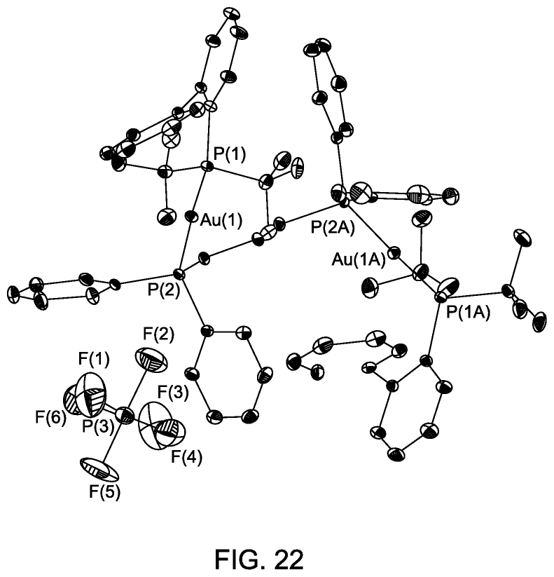

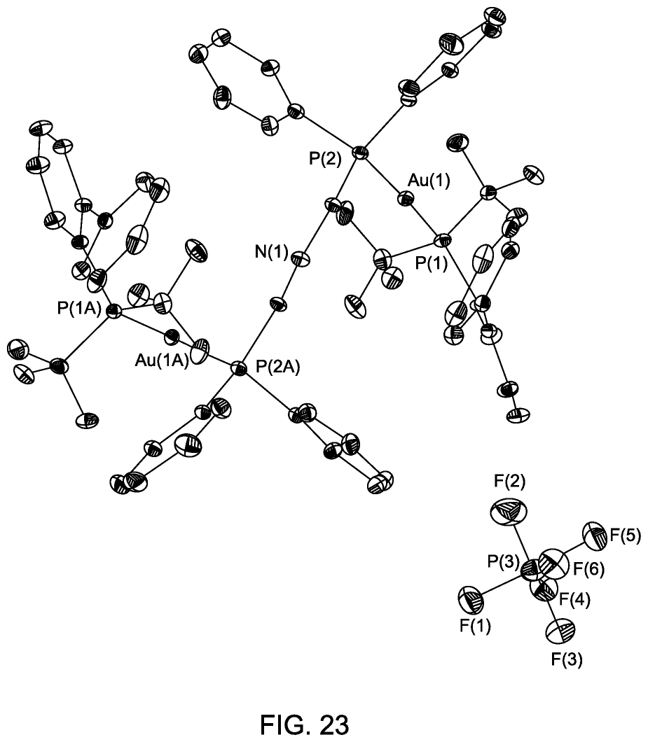

The X-ray crystal structures of complexes 1, 2, and 4 are shown in FIGS. 21-23, respectively. The selected bond parameters are given in Table 3. The structures of the complexes consist of cationic species: [Au₂(Bdpb)(Dppe)]²⁺ (complex 1), [Au₂(Bdpb)(Dppp)]²⁺ (complex 2), and [Au₂(Bdpb)(bdcpeaH)]²⁺ (complex 4), each with PF₆⁻ as counter ions. All three complexes are dinculear having each gold atom coordinated by one phosphorus atom of a bridging diphosphane ligand and one phosphorus atom of a terminal-bound mono-phosphane, (biphenyl-2-yl)di-tert-butylphosphane. The coordination geometry around the gold(I) atom is distorted linear with a (P—Au—P) bond angle of 170.79(4), 175.29(4), and 175.18(4) for 1, 2, and 4, respectively, which is seen in gold(I)-phosphane complexes. The phosphorus atoms of both phosphanes in the complexes possess a tetrahedral environment.

The Au—P bond lengths, which vary from 2.29(11) to 2.33(12) Å for the complexes, are similar to those reported for the analogous compounds, for example, 2.3013(13) to 2.3129(13) A in [{Au(Dppp)₂}₂]NiCl₄·(C₂H₅)₂O [A. Igashira-Kamiyama, T. Itai, Y. Arai, T. Konno, Bis[μ-1,3-bis(diphenylphosphanyl)propane-κ²P:P′]digold(I) tetrachloridonickelate(II) diethyl ether monosolvate, Acta Cryst. 2013, E69, m339, incorporated herein by reference in its entirety]. However, these Au—P distances in the complexes provided here within are shorter than in the tetrahedrally coordinated complex, [Au(Dppp)(PPh₃)Cl](2.39(13) Å) [F. Caruso, M. Rossi, J. Tanski, C. Pettinari, F. Marchetti, Antitumor Activity of the Mixed Phosphine Gold Species Chlorotriphenylposphine-1,3-bis(diphenylphosphino)propanegold(I). J. Med. Chem., 2003, 46, 1737-1742, incorporated herein by reference in its entirety]. The Au—P bond lengths in complex 4 are comparatively shorter than complexes 1-3 owing to the electron-donating nature of diphosphane. No Au—Au interactions were observed in the complexes. The complex cations and PF₆⁻ anions interact with each other through electrostatic interactions. No example of structures identical to those of 1, 2, and 4 could be found in the literature, although such complexes with chloride ions instead of tertiary phosphane are known [S. A. Bhat, J. T. Mague, M. S. Balakrishna, Dalton Trans. 2015, 44, 17696-17703. Gold(I) complexes of bisphosphines with bis(azol-lyl)methane backbone: Structure of a rare dinuclear gold(I) complex [(Au₂Cl){CH₂(1,2-C₃H2N₂PPh₂)₂}₃Cl]; and M. Streitberger, A. Schmied, E. Hey-Hawkins, Inorg. Chem. 2014, 53, 6794-6804. Selective Formation of Gold(I) Bis-Phospholane Macrocycles, Polymeric Chains, and Nanotubes, both of which are incorporated herein by reference in their entirety]. In a closely related complex, [{Au(PPh₃)}₂(μ₂-Dppe)Cl], the gold(I) atom is additionally coordinated with a chloride ion adopting a trigonal planar geometry [F. Caruso, C. Pettinari, F. Paduano, R. Villa, F. Marchetti, E. Monti, M. Rossi, J. Med. Chem. 2008, 51, 1584-1591. Chemical Behavior and in Vitro Activity of Mixed Phosphine Gold(I) Compounds on Melanoma Cell Lines, incorporated herein by reference in its entirety]. In [Au(PPh₃)(Dppp)Cl], the diphosphane ligand is bound in a chelating form, providing a tetrahedral environment around gold(I) [F. Caruso, M. Rossi, J. Tanski, C. Pettinari, F. Marchetti, Antitumor Activity of the Mixed Phosphine Gold Species Chlorotriphenylphosphine-1,3-bis(diphenylphosphino)propanegold(I). J. Med. Chem., 2003, 46, 1737-1742, incorporated herein by reference in its entirety].

TABLE 3
Selected bond lengths and bond angles for complexes 1, 2, and 4

| | |
|---|---|
| Bond Length (Å) | Bond Angles (°) |

Complex 1

| | | | |
|---|---|---|---|
| Au1-P1 | 2.2988(11) | P1-Au1-P2 | 170.79(4) |
| Au1-P2 | 2.3320(12) | C1-P1-Au1 | 115.23(17) |
| P1-C1 | 1.816(4) | C7-P1-Au1 | 116.13(15) |
| P1-C7 | 1.804(5) | C21-P2-Au1 | 116.51(16) |
| P2-C21 | 1.823(5) | C26-P2-Au1 | 106.3(2) |
| P2-C26 | 1.907(7) | C1-P1-C7 | 104.0(2) |
| F1-P3 | 1.523(6) | | |

Complex 2

| | | | |
|---|---|---|---|
| Au1-P1 | 2.3344(10) | P1-Au1-P2 | 175.29(4) |
| Au1-P2 | 2.3075(11) | C9-P2-C2 | 105.9(2) |
| P2-C9 | 1.810(4) | C9-P2-C3 | 107.7(2) |
| P2-C2 | 1.814(4) | C2-P2-C3 | 103.7(2) |
| P2-C3 | 1.816(5) | C9-P2-Au1 | 110.75(14) |
| P1-C15 | 1.820(5) | C2-P2-Au1 | 113.35(15) |
| P1-C32 | 1.877(5) | C3-P2-Au1 | 114.74(16) |

Complex 4

| | | | |
|---|---|---|---|
| Au1-P1 | 2.3297(16) | P2-Au1-P1 | 175.18(6) |
| Au1-P2 | 2.3052(15) | C1-P2-C21 | 105.9(3) |
| P2-C1 | 1.806(6) | C1-P2-C22 | 107.5(3) |
| P2-C21 | 1.821(6) | C21-P2-C22 | 103.9(3) |
| P2-C22 | 1.828(7) | C1-P2-Au1 | 110.9(2) |
| P1-C1 | 1.821(7) | C21-P2-Au1 | 113.4(2) |
| P1-C14 | 1.884(7) | C22-P2-Au1 | 114.6(2) |
| P1-C28 | 1.870(9) | C1-P1-C14 | 108.0(3) |

### Anti-Proliferative Activity of Complexes 1-4

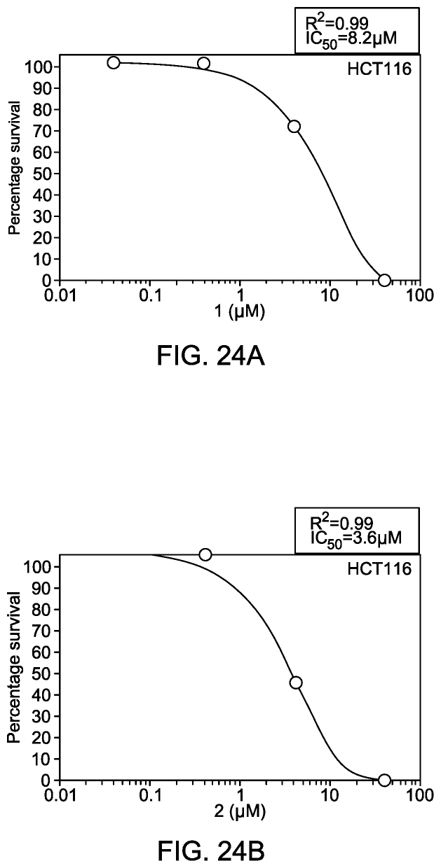

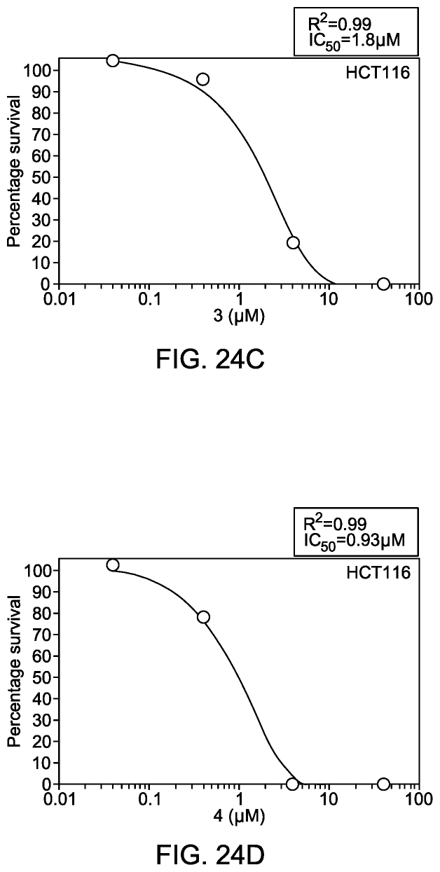

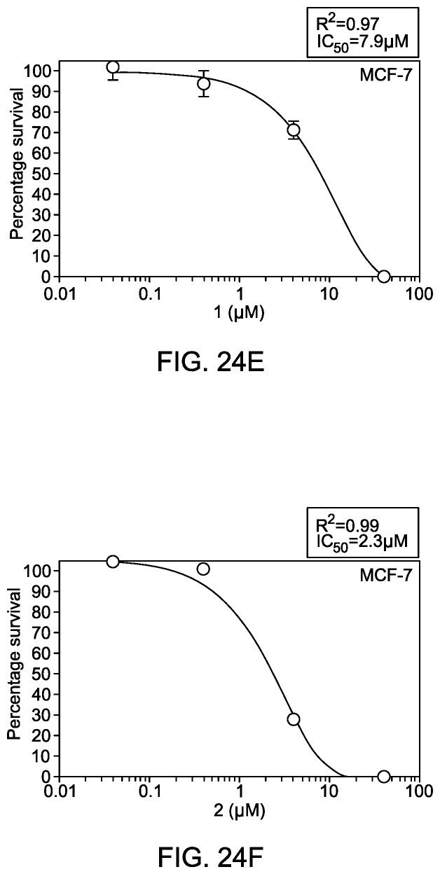

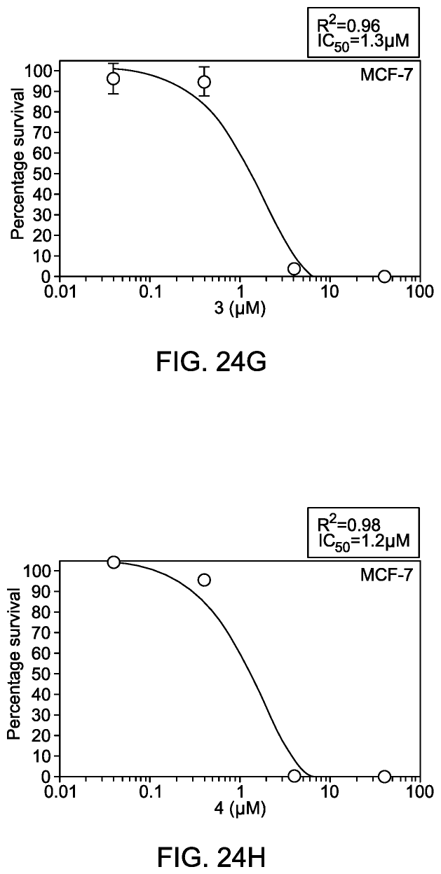

The prepared gold(J) complexes 1-4 were evaluated for their inhibition of cell proliferation towards two human cancer cell lines: HCT116 and MCF-7. The cells were treated with a range of concentrations (0.3, 1, 3, 10, 100 μM) of the complexes for 24 hours. The cells viability were then assessed and half maximal inhibitory concentration (IC₅₀) values were calculated thereafter. As shown in Table 4, the results have demonstrated that three of the four complexes displayed antiproliferative effects against both cancer cell models. Specifically, the IC₅₀ values for the complexes 1-4 were in the range of 0.93-8.2 μM for HCT116 cells, as can be observed in FIGS. 24A-24D, and 1.2-7.9 μM for MCF-7 cells, as can observed in FIGS. 24E-24H. These values are about 6- to 20-fold lower than cisplatin IC₅₀ values. Furthermore, the results showed that complex 4 had the highest antiproliferative capacity against both cell lines (IC₅₀=0.93 and 1.8 μM, respectively), while complex 1 had the least antiproliferative response (IC₅₀=8.2 μM and 7.9 μM, respectively).

Based on these findings, the presence of two gold atoms and lipophilic substituents in the complexes drive their anticancer activity. The data indicate that the length of the central linker has an effect on the potency (IC₅₀) of the complexes. Increasing the number of carbon atoms in the linker from 2 (complex 1) (FIG. 24A and FIG. 24E) to 4 (complex 3) (FIG. 24C and FIG. 24G) increased the cytotoxic effect of the complex by about 4.5-fold. The addition of a saturated benzene ring and amino group in the linker has increased the cytotoxicity of complex 4. These findings demonstrate modifications on a structure level that affect anticancer activity (structure-activity relationship).

TABLE 4
Half maximal inhibitory concentration (IC₅₀) values (μM) of gold(I)
complexes 1-4 against HCT116 and MCF-7 cancer cell lines

| | IC₅₀ (μM) | |
|---|---|---|
| Complex | HCT116 | MCF-7 |
| 1 | 8.20 ± 0.88 | 7.90 ± 3.07 |
| 2 | 3.60 ± 0.37 | 2.30 ± 0.24 |
| 3 | 1.80 ± 0.22 | 1.30 ± 0.54 |
| 4 | 0.93 ± 0.11 | 1.20 ± 0.32 |
| Cisplatin | 20.75 ± 0.93 | 21.06 ± 1.41 |
| [Au₂(dppm)(4-ep)₂] | — | 1.40 ± 0.20 [A. Meyer, A. Gutiérrez, I. Otta, L. |
| | | Rodríguez, Phosphine-bridged dinuclear gold(I) |
| | | alkynyl complexes: Thioredoxin reductase inhibition |
| | | and cytotoxicity, Inor. Chim. Acta 2013, 398, 72-76, |
| | | incorporated herein by reference in its entirety] |
| [Au₂(dppb)(4-ep)₂] | — | 2.8 ± 0.60 [A. Meyer, A. Gutiérrez, I. Otta, L. |
| | | Rodríguez, Phosphine-bridged dinuclear gold(I) |
| | | alkynyl complexes: Thioredoxin reductase inhibition |
| | | and cytotoxicity, Inor. Chim. Acta 2013, 398, 72-76, |
| | | incorporated herein by reference in its entirety] |
| [Au(dppp)(PPh₃)Cl] | 4.17 | [F. Caruso, M. Rossi, J. Tanski, C. Pettinari, F. |
| | | Marchetti, Antitumor Activity of the Mixed |
| | | Phosphine Gold Species Chlorotriphenylposphine- |
| | | 1,3-bis(diphenylphosphino) propanegold(I). J. Med. |
| | | Chem., 2003, 46, 1737-1742 F. Caruso, M. Rossi, J. |
| | | Tanski, C. Pettinari, F. Marchetti, Antitumor Activity |
| | | of the Mixed Phosphine Gold Species |
| | | Chlorotriphenylposphine- 1,3- |
| | | bis(diphenylphosphino)propanegold(I). J. Med. |
| | | Chem., 2003, 46, 1737-1742, incorporated herein by |
| | | reference in its entirety] |
| [(L1)₂Au]PF₆ | 1.75 | [C. Wetzel, P. C. Kunz, M. U. Kassack, A. |
| | | Hamacher, P. Bohler, W. Watjen, I. Ott, R. Rubbiani, |
| | | B. Spingler, Gold(I) complexes of water-soluble |
| | | diphos-type ligands: Synthesis, anticancer activity, |
| | | apoptosis and thioredoxin reductase inhibition, Dalton |
| | | Trans., 2011, 40, 9212, incorporated herein by |
| | | reference in its entirety] |
| [(L2)₂Au]Cl | 3.41 | [C. Wetzel, P. C. Kunz, M. U. Kassack, A. |
| | | Hamacher, P. Bohler, W. Watjen, I. Ott, R. Rubbiani, |
| | | B. Spingler, Gold(I) complexes of water-soluble |
| | | diphos-type ligands: Synthesis, anticancer activity, |
| | | apoptosis and thioredoxin reductase inhibition, Dalton |
| | | Trans., 2011, 40, 9212, incorporated herein by |
| | | reference in its entirety] |

4-ep = 4- ethynylpyridine
dppm = bis(diphenylphosphino)methane
dppb = 1,4-bis(diphenylphosphino)butane
dppp = 1,3-bis(diphenylphosphino)propane
L1 = Bis(1-methylimidazol-2-ylphenylphosphino)ethane
L2 = bis(dithiazol-2-ylphosphino)ethane

### Mitochondrial Potential Assay

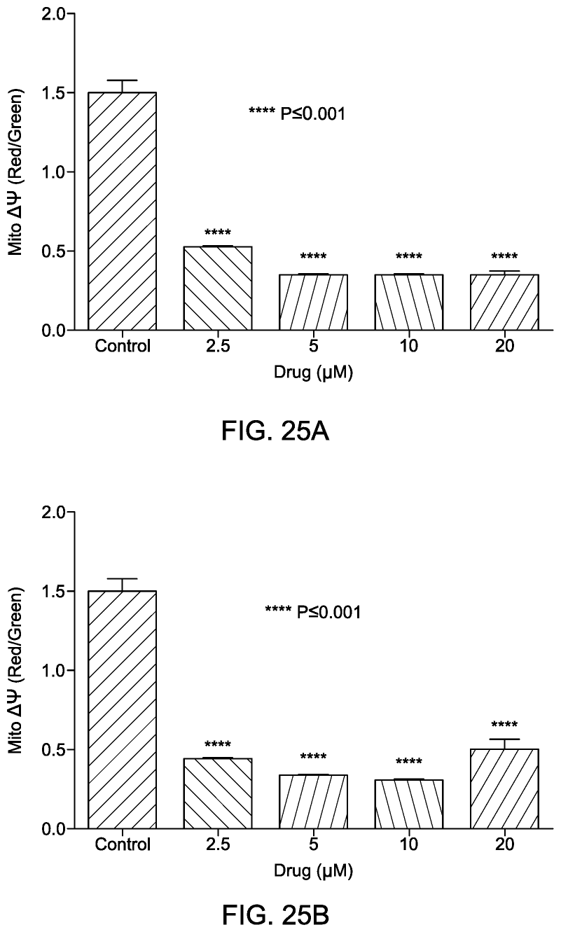

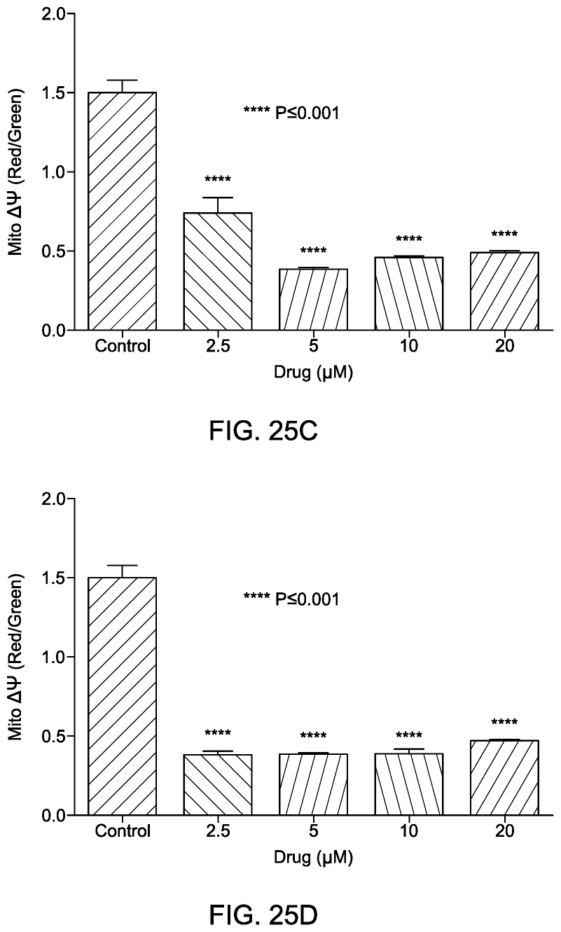

To examine the effect of complexes 1-4 on mitochondrial membrane potential (ΔΨm), JC-1 fluorescence dye was used to stain intact and depolarized mitochondria in MCF-7 cells following exposure to the complexes. The results showed that exposure to complexes 1-4 for 24 hours resulted in mitochondrial depolarization (FIG. 25A to FIG. 25D). Although all complexes resulted in severe mitochondrial depolarization (at 2.5 μM), the data shows that complex 4 was associated with the most reduction of ΔΨm, while complex 1 was associated with the least reduction of ΔΨm (FIG. 25 D and FIG. 25A, respectively). These findings are in accordance with the cytotoxicity studies (FIG. 24), indicating that these complexes interfere with mitochondrial health thereby leading to their anticancer activity.

Literature has demonstrated mitochondria as the primary site of the accumulation of gold-containing complexes. It is speculated that exposure to complexes 1-4 has an effective anticancer response mediated via oxidative stress-mediated activation of intrinsic apoptosis that is associated with disruption of the ΔΨm and potentially activating the intrinsic apoptotic pathway.

### Solution Chemistry of Complexes (2-4)

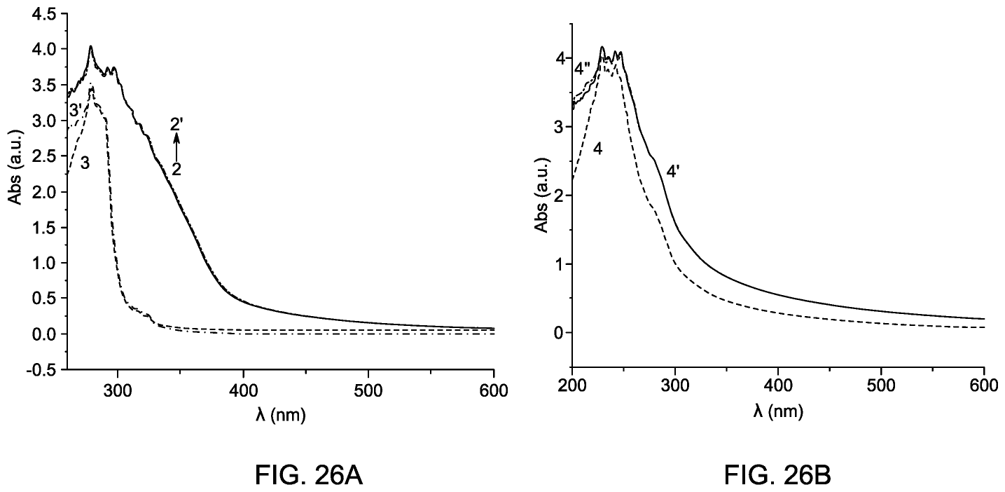

The solution chemistry of complexes 2, 3, and 4 (0.1 M, 10 mL) was investigated using ultraviolet-visible (UV-Vis) spectrophotometry at room temperature. The studied complexes are partially soluble in aqueous solution. Complexes 2 and 3 were dissolved in the mixture of DMSO:H₂O (3:7), where H₂O is a co-solvent of DMSO. A UV-Vis experiment was performed initially at time=0 (2 and 3) and at time=24 hours (2′ and 3′) (FIG. 26A). Furthermore, complex 4 was dissolved in 100% DMSO and a mixture of (DMSO:H₂O) (1:1), where H₂O is co-solvent of DMSO. Further, a UV-Vis experiment was performed initially at time=0 (4) and at time=24 hours. The results revealed that the studied complexes did not decompose or react with DMSO, indicating its high stability in 100% DMSO (4′) and a mixture of DMSO:H2O (4″) (FIG. 26B). Complex 3 showed less stability than complexes 2 and 4.

To conclude, the synthesis, spectral, and structural characterization and the anticancer activity of four dinuclear gold(I) complexes 1-4 containing a mono-phosphane and four different diphosphane ligands were evaluated. The crystal structures of complexes 1, 2, and 4 were determined by X-ray crystallography, which confirms that the complexes are dinuclear, exhibiting linear geometry at the gold centers. The in vitro cytotoxicity studies reveal that exposure to these gold(I) complexes is associated with inhibition of cell proliferation which is greater than that of the conventional chemotherapeutic agent cisplatin, as demonstrated in the viability studies against both HCT116 and MCF-7 human cancer cells. Furthermore, the data shows disruption of ΔΨm, indicating a potential activation of apoptotic cell death. Taken together, the findings demonstrate that gold(I) complexes provided herein represent candidates for anticancer therapy. The solution chemistry of complexes 2 and 4 indicated their stability in DMSO and a mixture of DMSO/H₂O. Complex 3 showed less stability in comparison with 2 and 4.

Numerous modifications and variations of the present disclosure are possible in light of the above teachings. It is therefore to be understood that within the scope of the appended claims, the invention may be practiced otherwise than as specifically described herein.

1: A gold complex, comprising:
a 2-(di-tert-butylphosphino)biphenyl ligand; and
a bis(diphenylphosphino)alkane ligand,
wherein the complex is dinuclear having two gold atoms,
wherein the 2-(di-tert-butylphosphino)biphenyl ligand and the bis(diphenylphosphino)alkane ligand are bonded to the gold atoms,
wherein the bis(diphenylphosphino)alkane ligand is bridging the two gold atoms.

2: The complex of claim 1, wherein the bis(diphenylphosphino)alkane ligand is selected from the group consisting of 1,2-bis(diphenylphosphino)ethane, 1,3-bis(diphenylphosphino)propane, 1,4-bis(diphenylphosphino)butane, and bis[2-(diphenylphosphino)methyl]amine.

3: The complex of claim 1, wherein the gold complex further comprises a hexafluorophosphate counterion.

4: The complex of claim 1, wherein a first phosphorous atom of the 2-(di-tert-butylphosphino)biphenyl ligand, a first gold atom, and a second phosphorous atom of the bis(diphenylphosphino)alkane ligand have a distorted linear geometry with a bond angle from 165° to 178°.

5: The complex of claim 1, having a half maximal inhibitory concentration of 0.5 to 10 μM in a first cancer cell line, HCT116.

6: The complex of claim 1, having a half maximal inhibitory concentration of 0.5 to 12 μM in a second cancer cell line, MCF-7.

7: The complex of claim 1, having a mitochondrial membrane potential of 0.2 to 0.8 in MCF-7 at a complex concentration of 2.5 μM compared to a mitochondrial membrane potential of 1.4 to 1.6 in MCF-7 in the absence of the complex.

8: The complex of claim 1, wherein the bis(diphenylphosphino)alkane ligand is bis[2-(diphenylphosphino)methyl]amine.

9: The complex of claim 8, having a half maximal inhibitory concentration of 0.6 to 1.2 μM in the first cancer cell line, HCT116.

10: The complex of claim 8, having a half maximal inhibitory concentration of 0.7 to 1.6 μM in the second cancer cell line, MCF-7.

11: The complex of claim 8, having a mitochondrial membrane potential of 0.2 to 0.5 in MCF-7 at a complex concentration of 2.5 μM compared to a mitochondrial membrane potential of 1.4 to 1.6 in MCF-7 in the absence of the complex.

12: The complex of claim 8, having a chemical stability in a solution of 100% by volume dimethyl sulfoxide (DMSO), 50% by volume DMSO and 50% by volume water, and 30% by volume DMSO and 70% by volume water based on a total volume of the solution.

13: The complex of claim 1, made by a process comprising:
mixing a silver hexafluorophosphate salt in a polar protic solvent with a (biphenyl-2-yl)di-tert-butylphosphane gold chloride salt in a polar aprotic solvent to form a reaction mixture;
filtering the reaction mixture to obtain a filtrate;
reacting the filtrate with the bis(diphenylphosphino) alkane ligand to form the complex,
wherein the silver hexafluorophosphate salt and the (biphenyl-2-yl)di-tert-butylphosphane gold chloride salt are in an amount double that of the molar amount of the bis(diphenylphosphino) alkane ligand.

14: A method for treating cancer, comprising:
administering the complex of claim 1 to a patient in need of treatment for cancer,
wherein during the administering the complex is contacted with an in vitro cancer cell line.

15: The method of claim 14, wherein administering the complex induces mitochondrial depolarization and apoptosis in MCF-7.

16: The method of claim 14, wherein the cancer is one or more of breast cancer, adenocarcinoma, breast adenocarcinoma, colon cancer, colorectal cancer, lung cancer, prostate cancer, kidney cancer, liver cancer, melanoma, pancreatic cancer, renal cancer, hepatocellular cancer, cervical cancer, and testicular cancer.

17: The method of claim 14, comprising administering the gold complex to a subject in a solution containing 0.1 to 150 μM of the gold complex.

18: The method of claim 14, wherein during the administering the complex is contacted with in vitro cancer cell line for 20 to 30 hours.

19: The method of claim 14, wherein the cancer cell line is cultured in a Dulbecco's Modified Eagle Medium (DMEM) with 5 to 15% by weight of a Fetal Bovine Serum (FBS) based on a total weight at 37° C. with 50 to 150 g/mL streptomycin and 50 to 150 units/mL penicillin.

20: A pharmaceutical composition comprising the complex of claim 1 or a pharmaceutically acceptable salt thereof, in combination with a pharmaceutically acceptable carrier, diluent, or excipient.
